SEQUENCE LISTING (24 sequences)

SEQ ID NO: 1 - protein, 374 aa, Feline herpesvirus 1
MMTRLHFWWCGIFAVLKYLVCTSSLTTTPKTTTVYVKGFNIPPLRYNYTQARIVPKIPQAMDPKITAEVRYVTSMDSCGMVALISEPDIDATIRTIQLSQKKTYNATISWFKVTQGCEYPMFLMDMRLCDPKREFGICALRSPSYWLEPLTKYMFLTDDELGLIMMAPAQFNQGQYRRVITIDGSMFYTDFMVQLSPTPCWFAKPDRYEEILHEWCRNVKTIGLDGARDYHYYWVPYNPQPHHKAVLLYWYRTHGREPPVRFQEAIRYDRPAIPSGSEDSKRSNDSRGESSGPNWIDIENYTPKNNVPIIISDDDVPTAPPKGMNNQSVVIPAIVLSCLIIALILGVIYYILRVKRSRSTAYQQLPIIHTTHHP

SEQ ID NO: 2 - protein, 948 aa, Feline herpesvirus 1
MSTRGDLGKRRRGSRWQGHSGYFRQRCFFPSLLGIAATGSRHGNGSSGLTRLARYVSFIWIVLFLVGPRPVEGQSGSTSEQPRRTVATPEVGGTPPKPTTDPTDMSDMREALRASQIEANGPSTFYMCPPPSGSTVVRLEPPRACPDYKLGKNFTEGIAVIFKENIAPYKFKANIYYKNIIMTTVWSGSSYAVTTNRYTDRVPVKVQEITDLIDRRGMCLSKADYVRNNYQFTAFDRDEDPRELPLKPSKFNTPESRGWHTTNETYTKIGAAGFHHSGTSVNCIVEEVDARSVYPYDSFAISTGDVIHMSPFFGLRDGAHVEHTSYSSDRFQQIEGYYPIDLDTRLQLGAPVSRNFLETPHVTVAWNWTPKSGRVCTLAKWREIDEMLRDEYQGSYRFTAKTISATFISNTSQFEINRIRLGDCATKEAAEAIDRIYKSKYSKTHIQTGTLETYLARGGFLIAFRPMISNELAKLYINELARSNRTVDLSALLNPSGETVQRTRRSVPSNQHHRSRRSTIEGGIETVNNASLLKTTSSVEFAMLQFAYDYIQAHVNEMLSRIATAWCTLQNREHVLWTETLKLNPGGVVSMALERRVSARLLGDAVAVTQCVNISSGHVYIQNSMRVTGSSTTCYSRPLVSFRALNDSEYIEGQLGENNELLVERKLIEPCTVNNKRYFKFGADYVYFEDYAYVRKVPLSEIELISAYVDLNLTLLEDREFLPLEVYTRAELEDTGLLDYSEIQRRNQLHALKFYDIDSIVRVDNNLVIMRGMANFFQGLGDVGAGFGKVVLGAASAVISTVSGVSSFLNNPFGALAVGLLILAGIVAAFLAYRYISRLRANPMKALYPVTTRNLKQTAKSPASTAGGDSDPGVDDFDEEKLMQAREMIKYMSLVSAMEQQEHKAMKKNKGPAILTSHLTNMALRRRGPKYQRLNNLDSGDDTETNLV

SEQ ID NO: 3 - protein, 534 aa, Feline herpesvirus 1
MRRYRMGRGIYLLYICLLYTYLQFGTSSTTAVSIENSDNSTAEMLSSTSMSATTPISQPTSPFTTPTRRSTNIATSSSTTQASQPTSTLTTLTRSSTTIATSPSTTQAATFIGSSTDSNTTLLKTTKKPKRKKNKNNGARFKLDCGYKGVIYRPYFSPLQLNCTLPTEPHITNPIDFEIWFKPRTRFGDFLGDKEDFVGNHTRTSILLFSSRNGSVNSMDLGDATLGILQSRIPDYTLYNIPIQHTEAMSLGIKSVESATSGVYTWRVYGGDGLNKTVLGQVNVSVVAYHPPSVNLTPRASLFNKTFEAVCAVANYFPPRSTKLTWYLDGKPIERQYISDTASVWIDGLITRSSVLAIPTTETDSEKPDIRCDLEWHESPVSYKRFTKSVAPDVYYPPTVSVTFADTRAICDVKCVPRDGISLMWKIGNYHLPKAMSADILITGPCIERPGLVNIQSMCDISETDGPVSYTCQTIGYPPILPGFYDTQVYDASPEIVSESMLVSVVAVILGAVLITVFIFITALCLYYSHPRRL

SEQ ID NO: 4 - DNA, 1125 bp, Feline herpesvirus 1
atgatgacccggctgcacttctggtggtgcggcatcttcgccgtgctgaaatacctggtgtgcacctccagcctgaccaccacccccaagaccaccaccgtgtacgtgaaaggcttcaacataccgccactgaggtacaactacacccaggccagaatcgtgcccaagatcccccaggctatggaccccaaaatcaccgccgaagtgagatacgtgaccagcatggactcttgcggcatggtggccctgatcagcgaacccgacatcgacgccaccatcagaaccatccagctgtctcagaagaaaacctacaacgccaccatctcctggttcaaggtgacccagggctgtgagtaccccatgttcctgatggacatgcggctgtgcgaccccaaacgcgagttcggcatctgtgccctgagaagcccctcttactggctggagcccctgaccaagtacatgttcctgaccgacgacgaactgggcctgatcatgatggcccccgcccagttcaaccagggccagtacaggagagtgatcaccatcgacggcagcatgttctacaccgacttcatggtgcagctgtctccaaccccatgctggttcgctaagccagaccggtacgaggaaatcctgcacgagtggtgtcgcaacgtgaaaaccatcggactggacggagctagggactaccactactactgggtgccctacaacccccagccccaccacaaagccgtgctgctgtactggtacaggacccacggcagggagccacccgtgcgcttccaggaagccatcagatacgacagacccgccatcccatccggcagcgaagactccaagaggagcaacgactctagaggcgagtcttccggccccaactggatcgacatcgaaaactacacccccaagaacaacgtgcccatcatcatctccgacgacgacgtgccaaccgctccacccaaaggcatgaacaaccagtccgtggtgatccccgccatcgtgctgagctgcctgatcatcgccctgatcctgggcgtgatctactacatcctgagggtgaagcggtctcgctccaccgcctaccagcagctgcccatcatccacaccacccaccacccctaa

SEQ ID NO: 5 - DNA, 1125 bp, Feline herpesvirus 1
atgatgacacgtctacatttttggtggtgtggaatctttgcggtcctgaaatatctggtatgtacttcaagccttacgaccacgccaaaaacaactacggtttatgtgaagggatttaatatacctccactacgctacaattatactcaagccagaatcgtgccaaaaattccccaggcgatggatccgaagataacagctgaagtacgttatgtaacatcaatggattcatgtgggatggtggcattgatatcagagccggatatagacgctactattcgaaccatacaactatctcaaaaaaaaacatataacgcgactataagttggtttaaggtaacccagggttgtgaataccctatgtttcttatggatatgagactttgtgatcctaaacgggaatttggaatatgtgctttacggtcgccttcatattggttggaacctttaacaaagtatatgttcctaacagacgatgaactgggtttgattatgatggccccggcccaatttaatcaaggacaatatcgaagagttataaccatcgatggttccatgttttatacagattttatggtacaactatctccaacgccatgttggttcgcaaaacccgatagatacgaagagattctacatgaatggtgtcgaaatgttaaaactattggccttgatggagctcgtgattaccactattattgggtaccctataacccacaacctcaccataaagccgtactcttatattggtatcggactcatggccgagaacccccagtaagattccaagaggccattcgatatgatcgtcccgccataccgtctgggagtgaggattcgaaacggtccaacgactctagaggagaatcgagtggacccaattggatagacattgaaaattacactcctaaaaataatgtgcctattataatatctgacgatgacgttcctacagcccctcccaagggcatgaataatcagtcagtagtgatacccgcaatcgtactaagttgtcttataatagcactgattctaggagtgatatattatattttgagggtaaagaggtctcgatcaactgcatatcaacaacttcctataatacatacaactcaccatccttaa

SEQ ID NO: 6 - DNA, 2847 bp, Feline herpesvirus 1
atgagcaccaggggcgacctgggcaagaggagacggggctccagatggcagggacacagcggatacttccggcagaggtgcttcttcccatccctgctgggaatcgctgctaccggcagcagacacggcaacggctccagcggactgaccaggctggccagatacgtgagcttcatctggatcgtgctgttcctggtgggaccaaggccagtggagggacagtctggctccaccagcgaacagccaaggaggaccgtggctaccccagaagtgggaggcaccccacccaagcccaccaccgaccccaccgacatgtccgacatgagggaggccctgagagcctctcagatcgaagccaacggcccctccaccttctacatgtgcccaccaccatctggctccaccgtggtgaggctggaaccacccagagcctgtcccgactacaagctgggcaaaaacttcaccgagggcatcgccgtgatcttcaaggaaaacatcgccccctacaagttcaaagccaacatctactacaaaaacatcatcatgaccaccgtgtggagcggctcttcctacgccgtgaccaccaacaggtacaccgaccgcgtgcccgtgaaggtgcaggagatcaccgacctgatcgacagacggggcatgtgcctgtccaaagccgactacgtgaggaacaactaccagttcaccgccttcgacagggacgaggacccccgcgaactgcccctgaagccatccaaattcaacacccccgagagcagaggctggcacaccaccaacgaaacctacaccaagatcggagctgctggattccaccacagcggcacctctgtgaactgtatcgtggaggaagtggacgccaggtccgtgtacccctacgactctttcgccatctccaccggcgacgtgatccacatgtcccccttcttcggactgagggacggagctcacgtggagcacaccagctacagctctgacagattccagcagatcgaaggctactaccccatcgacctggacacccggctgcagctgggagctcccgtgagccgcaacttcctggagaccccacacgtgaccgtggcttggaactggacccccaagtctggcagggtgtgcaccctggccaaatggcgggagatcgacgaaatgctgcgcgacgagtaccagggctcttacagattcaccgccaagaccatctccgccaccttcatctccaacaccagccagttcgaaatcaacaggatcagactgggcgactgtgctaccaaggaggctgctgaagccatcgacagaatctacaagtctaaatactccaaaacccacatccagaccggcaccctggagacctacctggccaggggcggcttcctgatcgccttcagacccatgatcagcaacgagctggccaaactgtacatcaacgaactggcccggtctaaccgcaccgtggacctgtctgccctgctgaacccatccggagaaaccgtgcagaggacccgcaggagcgtgccctctaaccagcaccacagatccagacggagcaccatcgagggcggcatcgaaaccgtgaacaacgccagcctgctgaagaccacctccagcgtggagttcgccatgctgcagttcgcctacgactacatccaggcccacgtgaacgaaatgctgtcccggatcgctaccgcttggtgcaccctgcagaacagggagcacgtgctgtggaccgaaaccctgaaactgaacccaggaggagtggtgagcatggccctggagcgcagggtgtctgccaggctgctgggcgacgctgtggctgtgacccagtgcgtgaacatctcttccggccacgtgtacatccagaactctatgcgcgtgaccggcagctctaccacctgttacagcaggcccctggtgtctttcagagccctgaacgactccgagtacatcgaaggccagctgggcgagaacaacgaactgctggtggagcggaagctgatcgaaccctgtaccgtgaacaacaagaggtacttcaagttcggcgccgactacgtgtacttcgaggactacgcctacgtgaggaaagtgcccctgagcgagatcgaactgatctctgcctacgtggacctgaacctgaccctgctggaggacagggagttcctgccactggaggtgtacacccgcgccgagctggaagacaccggactgctggactactctgaaatccagagacggaaccagctgcacgccctgaagttctacgacatcgactccatcgtgagggtggacaacaacctggtcatcatgagaggcatggccaacttcttccagggactgggcgacgtgggagctggattcggcaaagtggtgctgggagctgccagcgccgtgatctctaccgtgtccggcgtgtccagcttcctgaacaaccccttcggcgccctggccgtgggactgctgatcctggctggaatcgtggctgccttcctggcttacaggtacatcagccggctgagggctaacccaatgaaggccctgtaccccgtgaccacccgcaacctgaagcagaccgccaaaagccccgcctctaccgctggaggcgactccgaccccggagtggacgacttcgacgaggaaaagctgatgcaggcccgggagatgatcaaatacatgtccctggtgtccgctatggagcagcaggaacacaaggccatgaagaaaaacaaaggacccgccatcctgacctcccacctgaccaacatggccctgcgcaggagaggccccaagtaccagagactgaacaacctggacagcggcgacgacaccgagaccaacctggtgtaa

SEQ ID NO: 7 - DNA, 2847 bp, Feline herpesvirus 1
atgtccactcgtggcgatcttgggaagcggcgacgagggagtcgttggcagggacacagtggctattttcgacagagatgttttttcccttctctactcggtattgcagcgactggctccagacatggtaacggatcgtcgggattaaccagactagctagatatgtttcatttatctggatcgtactattcttagtcggtccccgtccagtagagggtcaatctggaagcacatcggaacaaccccggcggactgtagctacccctgaggtagggggtacaccaccaaaaccaactacagatcccaccgatatgtcggatatgagggaagctctccgtgcgtcccaaatagaggctaacggaccatcgactttttatatgtgtccaccaccttcaggatctactgtcgtgcgtttagagccaccacgggcctgtccagattataaactagggaaaaattttaccgagggtatagctgtaatatttaaagaaaatatagcgccatataaattcaaggcaaatatatactataaaaacattattatgacaacggtatggtctgggagttcctatgccgttacaaccaaccgatatacagacagggttcccgtgaaagttcaagagattacagatctcatagatagacggggtatgtgcctctcgaaagctgattacgttcgtaacaattatcaatttacggcctttgatcgagacgaggatcccagagaactgcctctgaaaccctccaagttcaacactccagagtcccgtggatggcacaccaccaatgaaacatacacaaagatcggtgctgctggatttcaccactctgggacctctgtaaattgcatcgtagaggaagtggatgcaagatctgtatatccatatgactcatttgctatctccactggtgacgtgattcacatgtctccattctttgggctgagggatggagcccatgtagaacatactagttattcttcagacagatttcaacaaatcgagggatactatccaatagacttggatacgcgattacaactgggggcaccagtttctcgcaattttttggaaactccgcatgtgacagtggcctggaactggaccccaaagtctggtcgggtatgtaccttagccaaatggagggaaatagatgaaatgctacgcgatgaatatcagggctcctatagatttacagccaagaccatatccgctactttcatctccaatacttcacaatttgaaatcaatcgtatccgtttgggggactgtgccaccaaggaggcagccgaagccatagaccggatttataagagtaaatatagtaaaactcatattcagactggaaccctggagacctacctagcccgtgggggatttctaatagctttccgtcccatgatcagcaacgaactagcaaagttatatatcaatgaattagcacgttccaatcgcacggtagatctcagtgcactcctcaatccatctggggaaacagtacaacgaactagaagatcggtcccatctaatcaacatcataggtcgcggcgcagcacaatagaggggggtatagaaaccgtgaacaatgcatcactcctcaagaccacctcatctgtggaattcgcaatgctacaatttgcctatgactacatacaagcccatgtaaatgaaatgttgagtcggatagccactgcctggtgtacacttcagaaccgcgaacatgtgctgtggacagagaccctaaaactcaatcccggtggggtggtctcgatggccctagaacgtcgtgtatccgcgcgcctacttggagatgccgtcgccgtaacacaatgtgttaacatttctagcggacatgtctatatccaaaattctatgcgggtgacgggttcatcaacgacatgttacagccgccctcttgtttccttccgtgccctcaatgactccgaatacatagaaggacaactaggggaaaacaatgaacttctcgtggaacgaaaactaattgagccttgcactgtcaataataagcggtattttaagtttggggcagattatgtatattttgaggattatgcgtatgtccgtaaagtcccgctatcggagatagaactgataagtgcgtatgtggatttaaatcttactctcctagaggatcgtgaatttctcccactcgaagtttatacacgagctgagctggaagataccggccttttggactacagcgagattcaacgccgcaaccaactccacgccttaaaattttatgatatagacagcatagtcagagtggataataatcttgtcatcatgcgtggtatggcaaatttttttcagggactcggggatgtgggggctggtttcggcaaggtggtcttaggggctgcgagtgcggtaatctcaacagtatcaggcgtatcatcatttctaaacaacccatttggagcattggccgtgggactgttaatattagctggcatcgtcgcagcattcctggcatatcgctatatatctagattacgtgcaaatccaatgaaagccttatatcctgtgacgactaggaatttgaaacagacggctaagagccccgcctcaacggctggtggggatagcgacccgggagtcgatgacttcgatgaggaaaagctaatgcaggcaagggagatgataaaatatatgtccctcgtatcggctatggagcaacaagaacataaggcgatgaaaaagaataagggcccagcgatcctaacgagtcatctcactaacatggccctccgtcgccgtggacctaaataccaacgcctcaataatcttgatagcggtgatgatactgaaacaaatcttgtctaa

SEQ ID NO: 8 - DNA, 1602 bp, Feline herpesvirus 1
atgaggagatacaggatgggcagaggcatctacctgctgtacatctgtctgctgtacacctacctgcagttcggcacctccagcaccaccgccgtgagcatcgagaactccgacaacagcaccgccgaaatgctgtcttccacctccatgagcgccaccaccccaatctcccagcccaccagccccttcaccaccccaacccggcgctccaccaacatcgccaccagctcttccaccacccaggcctctcagcccacctccaccctgaccaccctgacccggagctctaccaccatcgccacctctccctccaccacccaggctgccaccttcatcggctccagcaccgacagcaacaccaccctgctgaagaccaccaagaaacccaagcggaagaaaaacaaaaacaacggcgcccgcttcaagctggactgtggctacaaaggcgtgatctaccgcccctacttctcccccctgcagctgaactgcaccctgcccaccgagccccacatcaccaaccccatcgacttcgaaatctggttcaagccaaggacccgcttcggcgacttcctgggcgacaaagaggacttcgtgggcaaccacaccaggaccagcatcctgctgttctcttccagaaacggctctgtgaacagcatggacctgggcgacgccaccctgggcatcctgcagtctagaatccccgactacaccctgtacaacatccccatccagcacaccgaggccatgtccctgggcatcaagagcgtggaatctgccacctccggcgtgtacacctggagggtgtacggaggcgacggcctgaacaaaaccgtgctgggccaggtgaacgtgtctgtggtggcctaccacccaccatccgtgaacctgaccccaagagccagcctgttcaacaagaccttcgaggccgtgtgcgccgtggccaactacttcccccggagcaccaagctgacctggtatctggacggcaaacccatcgaacgccagtacatcagcgacaccgcctccgtgtggatcgacggactgatcaccaggagctctgtgctggccatccccaccaccgagaccgactccgaaaagcccgacatcaggtgcgacctggagtggcacgaaagccccgtgtcttacaagagattcaccaaaagcgtggctccagacgtgtactacccaccaaccgtgtctgtgaccttcgccgacacccgggctatctgcgacgtgaaatgcgtgccccgcgacggcatctccctgatgtggaagatcggcaactaccacctgcccaaagccatgagcgccgacatcctgatcaccggaccatgcatcgagagaccaggcctggtgaacatccagtccatgtgcgacatcagcgaaaccgacggccccgtgagctacacctgccagaccatcggctaccccccaatcctgccaggcttctacgacacccaggtgtacgacgcctctcccgagatcgtgagcgaatctatgctggtgtccgtggtggccgtgatcctgggagccgtgctgatcaccgtgttcatcttcatcaccgccctgtgcctgtactactcccaccccaggagattataa

SEQ ID NO: 9 - DNA, 1605 bp, Feline herpesvirus 1
atgagacgatataggatgggacgcggaatctaccttctctatatctgtctgttatatacatatctccagtttggtacttcgtcgacaaccgcggtcagtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattagattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatggactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaatcaatgttggttagtgtcgttgctgtaatactaggagctgttctcatcacagtctttatctttattacggcattatgtttatattattctcatccccggcgattataa

SEQ ID NO: 10 - DNA, 801 bp, Equine herpesvirus 1
atgttgactgtcttagcagccctgagtctgctcagcttgcttacgagcgcaaccggacggctcgccccagatgaactctgttatgccgaaccccgcagaactggcagcccaccaaacacccagcccgaacgcccacccgtaatatttgagcccccaacaattgcgattaaagctgaatccaagggttgtgagctaattttattagatccacccatagatgtaagctatcgcagagaagataaggtgaatgcgtccattgcttggttttttgactttggcgcttgccggatgcccatcgcatacagagagtattacggttgtattggcaatgctgttccctccccagagacttgtgatgcgtactcatttacccttattaggaccgagggtatcgtggagtttaccatcgtaaacatgagcctcctgtttcagcctggaatatacgatagtggcaattttatctacagcgttctcctggactaccacatatttacaggacgtgtaacgttggaagtggaaaaggacacaaactatccctgtggcatgattcatggactcactgcttacggaaacatcaacgtagatgaaaccatggacaacgccagcccacacccgcgtgccgtggggtgctttcccgagcccatcgacaacgaagcgtgggcaaacgttacatttactgaattggggataccagacccaaactcatttctcgatgacgagggtgattacccgaatatatcagactgtcactcgtgggagtcatacacctacccaaatacgctgaggcaggccacaggaccc

SEQ ID NO: 11 - DNA, 801 bp, Equine herpesvirus 1
atgttgactgtcttagcagctctgagtctgctcagcttgcttacgagcgcaaccggacggctcgccccagatgaactctgttatgccgaaccccgcagaactggcagcccaccaaacacccagcccgaacgcccacccgtaatatttgagcccccaacaattgcgattaaagctgaatccaagggttgtgagctaattttattagatccacccatagatgtaagctatcgcagagaagataaggtgaatgcgtccattgcttggttttttgactttggcgcttgccggatgcccatcgcatacagagagtattacggttgtattggcaatgctgttccctccccagagacttgtgatgcgtactcatttacccttattaggaccgagggtatcgtggagtttaccatcgtaaacatgagcctcctgtttcagcctggaatatacgatagtggcaattttatctacagcgttctcctggactaccacatatttacaggacgtgtaacgttggaagtggaaaaggacacaaactatccctgtggcatgattcatggactcactgcttacggaaacatcaacgtagatgaaaccatggacaacgccagcccacacccgcgtgccgtggggtgctttcccgagcccatcgacaacgaagcgtgggcaaacgttacatttactgaattggggataccagacccaaactcatttctcgatgacgagggtgattacccgaatatatcagactgtcactcgtgggagtcatacacctacccaaatacgctgaggcaggccacaggaccc

SEQ ID NO: 12 - DNA, 417 bp, Equine herpesvirus 1
ctccgagtaccccagaggagtatgtgaaaagctgccactcgcaactactgaagataatttcaacgctcaagataaatccggaggagtttcctcgagaccccgggtcgaggctcgtgcgcggatacatcgagtattctagactcgagcgcaagccctacacgcgctacccctgctttcaacgcgtcaacctgcacattgacggggagtttctggttcacaagatgctagcgttcaatgccgcgatgcgcccatcggccgaggagctgctgtcatacccaatgtttgctcaactttaggatgactaacctgtttctgggaggagacagcgtgggcgacggtgtataaagttggtctgctttcaagccctgccactgcgctacagtgccaccaactgtaaagcggtagtaagctgcagtg

SEQ ID NO: 13 - DNA, 431 bp, Equine herpesvirus 1
gaccctgttggtgggtgcggttggactcagaatcttggcgcaggcatggaagtttgtcggtgacgaaacatacgacaccatccgcgcagaagcaaagaatttagagacccacgtaccctcaagtgctgcagagtcgtctctagaaaaccaatcgacacaggaggagtctaacagccccgaagttgcccacctgcgaagcgtcaacagcgatgacagtacacacacggggggtgcgtcgaacggcatccaggactgtgacagtcagctcaaaactgtgtatgcctgcttggctctaattggactcggcacatgtgccatgatagggttgatagtttacatttgtgtattaaggtcaaaactgtcctctcggaatttttcgcgcgcgcaaaatgtaaaacatagaaattaccagcgacttgagtacgttgctt

SEQ ID NO: 14 - DNA, 417 bp, Equine herpesvirus 1
ctccgagtaccccagaggagtatgtgaaaagctgccactcgcaactactgaagataatttcaacgctcaagataaatccggaggagtttcctcgagaccccgggtcgaggctcgtgcgcggatacatcgagtattctagactcgagcgcaagccctacacgcgctacccctgctttcaacgcgtcaacctgcacattgacggggagtttctggttcacaagatgctagcgttcaatgccgcgatgcgcccatcggccgaggagctgctgtcatacccaatgtttgcacaactttaggatgactaacctgtttctgggaggagacagcgtgggcgacggtgtataaagttggtctgctttcaagccctgccactgcgctacagtgccaccaactgtaaagcggtagtaagctgcagtg

SEQ ID NO: 15 - DNA, 431 bp, Equine herpesvirus 1; identical to SEQ ID NO: 13

SEQ ID NO: 16 - DNA, 283 bp, Equine herpesvirus 1
tctagactcgagcgcaagccctacacgcgctacccctgctttcaacgcgtcaacctgcacattgacggggagtttctggttcacaagatgctagcgttcaatgccgcgatgcgcccatcggccgaggagctgctgtcatacccaatgtttgctcaactttaggatgactaacctgtttctgggaggagacagcgtgggcgacggtgtataaagttggtctgctttcaagccctgccactgcgctacagtgccaccaactgtaaagcggtagtaagctgcagtg

SEQ ID NO: 17 - DNA, 144 bp, Equine herpesvirus 1
gaccctgttggtgggtgcggttggactcagaatcttggcgcaggcatggaagtttgtcggtgacgaaacatacgacaccatccgcgcagaagcaaagaatttagagacccacgtaccctcaagtgctgcagagtcgtctctaga

SEQ ID NO: 18 - DNA, 430 bp, Equine herpesvirus 4
tctatttgaggacccgccgagtaccccacaagagtatgtaaaaagctgtcattctcaactactgagaataatatcaaagctaaagataaaccctgaggagtttccacgggaaccagagtctaggctcgtgcgcggatacatcgaatacgccagcctagagcgtaagccacatacgcgctatccttgcttccagcgcgtgaacctacacattgacggggaatttttgatccataaaatgctagcgttcaatgctgcgatgcgcccatccgcagaagagttgttgtcctacccaatgtttatgaatctgtaggatgactaacagatttggggtggagacggcgtgggcgatactgtataaagttgtactacttaccagcccagtcagtgtgctgtagtgccaccacctgtaaagctgtgataagctgcagtt

SEQ ID NO: 19 - DNA, 449 bp, Equine herpesvirus 4
ttggtggtagcatatactaccttatttatacgctccgagctgtttttcagcatgctagcacccaacgccgagcgagagtatataactcccatcattgcccacaagcttatgccacttattagcgtccgctctgccgtttgcttagtcataatatctaccgccgtttacgcagcagacgctatctgcgacacaattggatttgcgataccgcgcatgtggatgtgtattttaatgagatcaacctccatgaagcgtaactagggggcctcccactgaggcactaccggcttagcagctgactaacacagtataaaacgtgagaagaaatcagtctcatgcgccattagcgctaggctagttagcgtggaggaccggagcgctaccgccagcagtttcatccgcctggttacgggtttgttaacacctaccggtgttttaccgctaccata

SEQ ID NO: 20 - DNA, 100 bp, Artificial Sequence
  Primer
cccaggtcgcccctggtgctcatgggtcctgggttggactccacgtcgccagccagcttcagcaggtcgaagttcagggggtggtgggtggtgtggatga

SEQ ID NO: 21 - DNA, 25 bp, Artificial Sequence
  Primer
gactctagaggcgagtcttccggcc

SEQ ID NO: 22 - DNA, 23 bp, Artificial Sequence
  Primer
atgagcaccaggggcgacctggg

SEQ ID NO: 23 - DNA, 36 bp, Artificial Sequence
  Primer
ctatgtcgacctcgagggtaccttacaccaggttgg

SEQ ID NO: 24 - DNA, 400 bp, Feline herpesvirus 1
ctcataagttttttgagctggtgaacgggcctctattcgaccacgacagtcataactttgcccaaccccccaacacagcgttttatttcagtgttgagaacgttggtctgcttccacatttaaaagaagaattggcgggatttatgttaacgtccacccggggtgggtggacggtgagtaaatttcaaagattttactatttcggtgatgatacgtctggcgtcacaacaactcagcggttggcttggaaatatatccgtgagctcattctagcatctgccatattttcctccgtgtttcactgcggtgaggtgaagttgctacgctctgatcgcacacgaccggctaatacaggtacccagatctgcccacccggcatttatctaacatacgaagaatc

(19) United States
(12) Patent Application Publication
(10) Pub. No.: US 2025/0249090 A1
(43) Pub. Date: Aug. 7, 2025

(54) NEW FELINE HERPES VIRUS VACCINE

(71) Applicant: Boehringer Ingelheim Vetmedica GmbH, Ingelheim am Rhein (DE)

(72) Inventors: Callie Ann Visek, Ames, IA (US); Robert Barry Mandell, Collins, IA (US); Teshome Mebatsion, Acworth, GA (US); Ramesh Koukuntla, Cypress, TX (US); Eric Martin Vaughn, Ames, IA (US)

(21) Appl. No.: 18/252,590

(22) PCT Filed: Nov. 11, 2021
(86) PCT No.: PCT/IB2021/000814

§ 371 Date: May 11, 2023

Related U.S. Application Data
(60) Provisional application No. 63/113,398, filed on Nov. 13, 2020.

(51) Int. Cl. A61K 39/245; A61K 39/00; A61P 37/04

(52) U.S. Cl. CPC A61K 39/245; A61P 37/04; A61K 2039/5256; A61K 2039/552

(57) ABSTRACT

The present invention relates i.a. to an EHV (Equine Herpesvirus) comprising a Feline Herpes Virus (FHV) Antigen encoding sequence inserted into ORF70 (US4) and/or ORF1/3. Furthermore, the present invention relates to methods for immunizing a feline comprising administering to such feline an immunogenic composition of the present invention. Moreover, the present invention relates to methods for the treatment or prophylaxis of clinical signs caused by Feline Herpes Virus in a feline.

### SEQUENCE LISTING

The instant application contains a Sequence Listing which has been submitted electronically in .TXT file format and is hereby incorporated by reference in its entirety. Said .TXT copy, created on Mar. 3, 2023, is named 01_3463 Priosequence listing_ST25_SEQ and is 43 kilobytes in size.

### BACKGROUND OF THE INVENTION

### A. Field of the Invention

The present invention relates to the field of Feline Herpes Virus Vaccines.

### B. Background and Description of the Related Art

### Feline Herpes Virus (FHV)

FHV is the causative agent of feline viral rhinotracheitis in cats, the disease is found world-wide. Feline herpesvirus causes upper respiratory infection with rhinitis, fever, sneezing, eye and nasal discharge, conjunctivitis (inflammation of the inner eyelids and mucous membranes around the eyes), inflammation of the cornea (keratitis), and lethargy.

Modified live as well as killed FHV vaccines have been developed.

However, in general modified live vaccine strains may revert to a virulent state resulting in disease of the inoculated animals and the possible spread of the pathogen to other animals.

Inactivated vaccines generally induce only a low level of immunity (no or only weak stimulation of the cellular immunity).

In contrast, vector vaccines may induce both FHV neutralizing antibodies and a cellular immunity against FHV.

Another issue arises by the fact that FHV cultivation for vaccine production takes place in feline kidney cell lines. However, vaccines that contain residual components of said cells may cause cats to generate autoantibodies causing chronic kidney disease or other diseases. Therefore, there is a need for FHV vaccines that have not been cultivated in feline originating cell lines.

This problem of autoantibodies is already discussed in the literature such as in Lappin et al 2005 (AJVR, Vol 66, No. 3), Whittemore et al 2010 (J Vet Intern Med;24:306-313) and Songaksorn et al 2019 (Vet Sci. 2019 November;20(6):e73) describing that the Crandell feline kidney (CRFK) cell line is used to propagate feline viruses such as feline herpesvirus 1 (FHV-1), calicivirus, and panleukopenia virus and that during vaccine production it would be impossible to remove all CRFK cell proteins or other cell constituents. As a consequence, during the course of routine vaccination, cats are exposed to CRFK cell proteins and develop an immune response against those proteins. The study results shown in Lappin et al 2005 and Whittemore et al 2010 describe that administration of vaccines containing viruses grown on CRFK cells induced antibodies in cats such as autoantibodies to kidney tissue.

Furthermore, with currently administered live attenuated or inactivated FHV vaccines it is not possible to determine whether a specific animal is a carrier of an FHV field virus or whether the animal was vaccinated. Thus, both modified live vaccines and inactivated vaccines lack the inherent feature for the diagnostic differentiation of infected from vaccinated animals (DIVA). Thus, there is a need for FHV DIVA vaccines.

For the above mentioned reasons there is a need for new Feline Herpes virus vaccines.

### DETAILED DESCRIPTION OF THE INVENTION

Before the aspects of the present invention are described, it must be noted that as used herein and in the appended claims, the singular forms “a”, “an”, and “the” include plural reference unless the context clearly dictates otherwise. Thus, for example, reference to “an antigen” includes a plurality of antigens, reference to the “virus” is a reference to one or more viruses and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies as reported in the publications which might be used in connection with the invention. Nothing herein is to be construed as an admission that the invention is not entitled to antedate such disclosure by virtue of prior invention.

The present invention solves the problems inherent in the prior art and provides a distinct advance in the state of the art. Generally, the present invention provides an EHV (Equine Herpesvirus) comprising a Feline Herpes Virus (FHV) Antigen encoding sequence inserted into ORF70 (US4) and/or ORF1/3.

Further, the present invention provides an EHV (Equine Herpesvirus) comprising one single Feline Herpes Virus (FHV) Antigen encoding sequence inserted into ORF70 (US4) and/or one single Feline Herpes Virus (FHV) Antigen encoding sequence inserted into ORF1/3.

Furthermore, the present invention provides an EHV (Equine Herpesvirus) comprising a Feline Herpes Virus (FHV) Antigen encoding sequence encoding equal or less than 1400 amino acids inserted into ORF70 (US4) and/or ORF1/3.

Advantageously, the experimental data provided by the present invention disclose that when the Feline Herpes Virus (FHV) Antigen is inserted into EHV using the ORF70 (US4) and/or ORF1/3 insertion site, the antigen is well expressed. Thus, the EHV when using the ORF70 (US4) and/or ORF1/3 insertion site is suitable for expressing FHV antigens.

The term “EHV” means Equine Herpes virus or horse pathogen Equid Alphaherpesvirus (Equine abortion virus) and belongs to the genus Varicellovirus in the subfamily Alphaherpesvirinae in the family Herpesviridae in the order Herpesvirales.

The term “ORF70” or “US4” defines an insertion site within EHV that is well known to a person skilled in the art and already has been described in the prior art such as in WO 2018/054837 A1. An insertion in ORF70 means that a DNA fragment was inserted into the genomic DNA at a location encoding the Equid herpesvirus 1 open reading frame 70 abolishing expression of the orf70 gene product glycoprotein G. However, preferably the ORF71 remains functional or intact.

The term “ORF1/3” defines another insertion site within EHV that is well known to a person skilled in the art and already has been described in the prior art such as in WO 2018/054837 A1 or Said et al. 2013 (Virus Research 173: 371-376). When using this insertion site, ORF1/ORF2 are affected and, thus, the insertion site is also called ORF1/ORF2 insertion site. Originally, this insertion site was identified by coincidence. It was observed that by accidental deletion over passaging during the attenuation procedure of the vaccine strain EHV-1 RacH a 1283 bp fragment comprising 90% of ORF1 and the entire ORF2 was lost.

In general, “open reading frame” or “ORF” refers to a length of nucleic acid sequence, either DNA or RNA that comprises a translation start signal or initiation codon, such as an ATG or AUG, and a termination codon and can be potentially translated into a polypeptide sequence.

An “antigen” as used herein refers to, but is not limited to, components which elicit an immunological response in a host to an immunogenic composition or vaccine of interest comprising such antigen or an immunologically active component thereof. The term “antigen” encompasses full length proteins as well as peptide fragments thereof containing or comprising one or more epitopes. Further, the term “antigen encoding sequence” relates to sequences encoding an antigen. Preferably, the antigen encoding sequence is a nucleic acid sequence such as a cDNA or DNA sequence. However, the term “nucleic acid sequence” has been defined elsewhere herein.

The term “Feline Herpes Virus” or “FHV” is well known to the person skilled in the art. FHV is the causative agent of feline viral rhinotracheitis in cats and causes upper respiratory infection. FHV belongs to the family Herpesviridae, subfamily Alphaherpesvirinae. Several complete FHV genomes were already sequenced such as wild-type strain C-27, the sequence is publicly available (ACCN: FJ478159). Preferably, the FHV is FHV-1.

### Insertion Sites

In one specific aspect of the EHV according to the present invention the Feline Herpes Virus (FHV) Antigen encoding sequence is inserted into ORF70 (US4).

In another specific aspect of the EHV according to the present invention the Feline Herpes Virus (FHV) Antigen encoding sequence is inserted into ORF1/3.

In another specific aspect of the EHV according to the present invention the Feline Herpes Virus (FHV) Antigen encoding sequence is inserted into ORF70 (US4) and ORF1/3.

### Length of Feline Herpes Virus (FHV) Antigen

In another specific aspect of the EHV according to the present invention the FHV Antigen encoding sequence encodes equal or less than 1400 amino acids.

Advantageously, the experimental data provided by the present invention disclose that when the Feline Herpes Virus (FHV) Antigen is inserted into EHV using the ORF70 (US4) and/or ORF1/3 insertion site, the antigen is well expressed.

In another specific aspect of the EHV according to the present invention the FHV Antigen encoding sequence encodes equal or less than 1200 amino acids.

In another specific aspect of the EHV according to the present invention the FHV Antigen encoding sequence encodes equal or less than 1000 amino acids.

In another specific aspect of the EHV according to the present invention the FHV Antigen encoding sequence encodes between 200 and 1200 amino acids.

In another specific aspect of the EHV according to the present invention the FHV Antigen encoding sequence encodes between 200 and 1000 amino acids.

The term “protein”, “amino acid” and “polypeptide” are used interchangeably. The term “protein” refers to a sequence of amino acids composed of the naturally occurring amino acids as well as derivatives thereof. The naturally occurring amino acids are well known in the art and are described in standard text books of biochemistry. Within the amino acid sequence the amino acids are connected by peptide bonds. Further, the two ends of the amino acid sequence are referred to as the carboxyl terminus (C-terminus) and the amino terminus (N-terminus). The term “protein” encompasses essentially purified proteins or protein preparations comprising other proteins in addition. Further, the term also relates to protein fragments. Moreover, it includes chemically modified proteins. Such modifications may be artificial modifications or naturally occurring modifications such as phosphorylation, glycosylation, myristylation and the like.

In another specific aspect of the EHV according to the present invention the FHV Antigen encoding sequence comprises equal or less than 4200 nucleic acids.

In another specific aspect of the EHV according to the present invention the FHV Antigen encoding sequence comprises equal or less than 3600 nucleic acids.

In another specific aspect of the EHV according to the present invention the FHV Antigen encoding sequence comprises equal or less than 3000 nucleic acids.

In another specific aspect of the EHV according to the present invention the FHV Antigen encoding sequence encodes between 600 and 3600 nucleic acids.

In another specific aspect of the EHV according to the present invention the FHV Antigen encoding sequence encodes between 600 and 3000 nucleic acids.

The term “nucleic acid” or “nucleic acid sequence” or “nucleotide sequence” refers to polynucleotides including DNA molecules, RNA molecules, cDNA molecules or derivatives. The term encompasses single as well as double stranded polynucleotides. The nucleic acid of the present invention encompasses isolated polynucleotides (i.e. isolated from its natural context) and genetically modified forms. Moreover, comprised are also chemically modified polynucleotides including naturally occurring modified polynucleotides such as glycosylated or methylated polynucleotides or artificially modified ones such as biotinylated polynucleotides. Further, the terms “nucleic acid” and “polynucleotide” are interchangeable and refer to any nucleic acid. The terms “nucleic acid” and “polynucleotide” also specifically include nucleic acids composed of bases other than the five biologically occurring bases (adenine, guanine, thymine, cytosine and uracil).

### Definition of Antigen and gD, gB and gC

In another specific aspect of the EHV according to the present invention the FHV Antigen encoding sequence is one FHV Antigen encoding sequence.

In another specific aspect of the EHV according to the present invention the FHV Antigen encoding sequence is one single FHV Antigen encoding sequence.

In another specific aspect of the EHV according to the present invention the FHV Antigen encoding sequence is not two or more FHV Antigen encoding sequences.

Advantageously, the experimental data provided by the present invention disclose that when the Feline Herpes Virus (FHV) Antigen is inserted into EHV using the ORF70 (US4) and/or ORF1/3 insertion site, the antigen is well expressed. It has been shown that the expression is superior when only one FHV antigen is inserted per insertions site compared to gD and gB with a F2A in-between inserted at the ORF1/3 site (as shown in Example 6, Table 5, 7 and 8).

Preferably, the FHV antigen is a glycoprotein. The herpesvirus antigen may be a glycoprotein B (gB), glycoprotein C (gC) or glycoprotein D (gD) antigen from a feline herpesvirus.

In another specific aspect of the EHV according to the present invention the FHV Antigen encoding sequence is a glycoprotein encoding sequence or a fragment thereof.

In another specific aspect of the EHV according to the present invention the FHV Antigen encoding sequence is a FHV gD (glycoprotein D) encoding sequence or a fragment thereof and/or a FHV gB (glycoprotein B) encoding sequence or a fragment thereof and/or a FHV gC (glycoprotein C) encoding sequence or a fragment thereof.

The Herpesvirus glycoprotein D (gD) is essential for FHV-1 (Feline Herpesvirus-1) entry and is involved in interaction with host cell (binding to receptors). The gD protein has haemagglutination activities on feline red blood cells. The Herpesvirus glycoprotein B (gB) is essential for FHV entry and is involved in fusion process. Both glycoproteins can induce neutralizing antibodies. The Herpesvirus glycoprotein C (gC) is involved in viral attachment to cell through interactions with heparin sulfate. Glycoprotein gC can induce neutralizing antibodies as well.

In another specific aspect of the EHV according to the present invention said FHV Antigen encoding sequence is a FHV gD encoding sequence or a fragment thereof.

In another specific aspect of the EHV according to the present invention said FHV Antigen encoding sequence is a FHV gB encoding sequence or a fragment thereof.

In another specific aspect of the EHV according to the present invention said FHV Antigen encoding sequence is a FHV gC encoding sequence or a fragment thereof.

### Fragments of Glycoproteins

In another specific aspect of the EHV according to the present invention said fragment of the glycoprotein encoding sequence encodes at least 100, at least 150, at least 200, at least 300, at least 350, at least 400, at least 500, at least 600, at least 700 or at least 800 amino acids.

In another specific aspect of the EHV according to the present invention said fragment of the glycoprotein encoding sequence comprises at least 300, at least 450, at least 600, at least 900, at least 1050, at least 1200, at least 1500, at least 1800, at least 2100 or at least 2400 nucleic acids.

In another specific aspect of the EHV according to the present invention said fragment of the glycoprotein encoding sequence encodes at least 100, at least 200, at least 300 or at least 350 amino acids.

In another specific aspect of the EHV according to the present invention said fragment of the glycoprotein encoding sequence comprises at least 300, at least 600, at least 900 or at least 1050 nucleic acids.

In another specific aspect of the EHV according to the present invention said fragment of the FHV gD encoding sequence encodes of at least 100, at least 200, at least 250, at least 300 or at least 350 amino acids.

In another specific aspect of the EHV according to the present invention said fragment of the FHV gD encoding sequence comprises at least 300, 600, 750, 900 or 1050 nucleic acids.

In another specific aspect of the EHV according to the present invention said fragment of the FHV gB encoding sequence encodes at least 100, at least 150, at least 200, at least 300, at least 400, at least 500, at least 600, at least 700 or at least 800 amino acids.

In another specific aspect of the EHV according to the present invention said fragment of the FHV gB encoding sequence comprises at least 300, at least 450, at least 600, at least 900, at least 1200, at least 1500, at least 1800, at least 2100 or at least 2400 nucleic acids.

In another specific aspect of the EHV according to the present invention said fragment of the FHV gC encoding sequence encodes at least 100, at least 150, at least 200, at least 300, at least 400 or at least 500 amino acids.

In another specific aspect of the EHV according to the present invention said fragment of the FHV gC encoding sequence comprises at least 300, at least 450, at least 600, at least 900, at least 1200 or at least 1500 nucleic acids.

### Definition of gB, gC and gD by Sequence

In another specific aspect of the EHV according to the present invention said FHV Antigen encoding sequence is a FHV gD encoding sequence and/or a FHV gB encoding sequence and/or a FHV gC encoding sequence based on wild-type feline herpesvirus strain C-27 (ACCN: FJ478159).

In another specific aspect of the EHV according to the present invention said FHV Antigen encoding sequence is a FHV gD encoding sequence consisting of or comprising a nucleic acid sequence encoding the amino acid sequence as shown in AAB30980.1 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

The term “identity” or “sequence identity” is known in the art and refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

The terms “identity”, “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are of the same length.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

The skilled person will be aware of the fact that different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

The protein sequences or nucleic acid sequences of the present invention can further be used as a “query sequence” to perform a search against public databases to, for example, to identify other family members or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

As used herein, it is in particular understood that the term “identical to the sequence of SEQ ID NO: X” is equivalent to the term “identical to the sequence of SEQ ID NO: X over the length of SEQ ID NO: X” or to the term “identical to the sequence of SEQ ID NO: X over the whole length of SEQ ID NO: X”, respectively. In this context, “X” is any integer selected from 1 to 24 so that “SEQ ID NO: X” represents any of the SEQ ID NOs mentioned herein.

In another specific aspect of the EHV according to the present invention said FHV Antigen encoding sequence is a FHV gB encoding sequence consisting of or comprising a nucleic acid sequence encoding the amino acid sequence as shown in AAB28559.3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids.

In another specific aspect of the EHV according to the present invention the FHV Antigen encoding sequence is a glycoprotein encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In another specific aspect of the EHV according to the present invention the FHV Antigen encoding sequence is a gD, gB or gC encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In another specific aspect of the EHV according to the present invention the FHV Antigen encoding sequence is a glycoprotein consisting of or comprising a nucleic acid sequence as set forth in SEQ ID NO:4, SEQ ID NO:5, SEQ ID NO:6, SEQ ID NO:7, SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In another specific aspect of the EHV according to the present invention the FHV Antigen encoding sequence is a gD, gB or gC consisting of or comprising a nucleic acid sequence as set forth in SEQ ID NO:4, SEQ ID NO:5, SEQ ID NO:6, SEQ ID NO:7, SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In another specific aspect of the EHV according to the present invention said gD encoding sequence consists of or comprises a nucleic acid sequence encoding an amino acid sequence as set forth in SEQ ID NO:1 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In another specific aspect of the EHV according to the present invention said gD encoding sequence consists of or comprises a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In another specific aspect of the EHV according to the present invention said gB encoding sequence consists of or comprises a nucleic acid sequence encoding an amino acid sequence as set forth in SEQ ID NO:2 or a sequence having at least 70%, at least 75%, at least 80%, at least 85%, at least 90%, at least 93%, at least 95%, at least 96%, at least 97%, at least 98%, at least 99%, 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids.

In another specific aspect of the EHV according to the present invention said gB encoding sequence consists of or comprises a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In another specific aspect of the EHV according to the present invention said gC encoding sequence consists of or comprises a nucleic acid sequence encoding an amino acid sequence as set forth in SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400 or at least 500 consecutive amino acids.

In another specific aspect of the EHV according to the present invention said gC encoding sequence consists of or comprises a nucleic acid sequence encoding an amino acid sequence as set forth in SEQ ID NO:3 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In another specific aspect of the EHV according to the present invention said gD encoding sequence consists of or comprises a nucleic acid sequence as set forth in SEQ ID NO:4 or SEQ ID NO:5 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In another specific aspect of the EHV according to the present invention said gD encoding sequence consists of or comprises a nucleic acid sequence as set forth in SEQ ID NO:4 or SEQ ID NO:5 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In another specific aspect of the EHV according to the present invention said gB encoding sequence consists of or comprises a nucleic acid sequence as set forth in SEQ ID NO:6 or SEQ ID NO:7 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 450, at least 600, at least 900, at least 1200, at least 1500, at least 1800, at least 2100 or at least 2400 consecutive nucleic acids.

In another specific aspect of the EHV according to the present invention said gB encoding sequence consists of or comprises a nucleic acid sequence as set forth in SEQ ID NO:6 or SEQ ID NO:7 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In another specific aspect of the EHV according to the present invention said gC encoding sequence consists of or comprises a nucleic acid sequence as set forth in SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 450, at least 600, at least 900, at least 1200 or at least 1500 consecutive nucleic acids.

In another specific aspect of the EHV according to the present invention said gC encoding sequence consists of or comprises a nucleic acid sequence as set forth in SEQ ID NO:8 or SEQ ID NO:9 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

### EHV

In another specific aspect of the EHV according to the present invention the EHV is a vector.

In another specific aspect of the EHV according to the present invention the EHV is an EHV-vector.

In another specific aspect of the EHV according to the present invention the EHV is attenuated.

The term “attenuated” refers to a pathogen having a reduced virulence in comparison to the wildtype isolate. In the present invention “attenuation” is synonymous with “avirulent”. In the present invention, an attenuated virus is one in which the virulence has been reduced so that it does not cause clinical signs of infection but is capable of inducing an immune response in the target animal, but may also mean that the clinical signs are reduced in incidence or severity in animals infected with the attenuated virus, especially the EHV-1 RacH viral vector, in comparison with a “control group” of animals infected with non-attenuated virus or pathogen and not receiving the attenuated virus. In this context, the term “reduce/reduced” means a reduction of at least 10%, preferably 25%, even more preferably 50%, still more preferably 60%, even more preferably 70%, still more preferably 80%, even more preferably 90% and most preferably of 100% as compared to the control group as defined above.

In another specific aspect of the EHV according to the present invention the EHV is genetically engineered.

The term “genetically engineered” refers to an EHV which has been mutated by using “reverse genetics” approaches. Preferably, the EHV according to the present invention has been genetically engineered. However, “reverse genetics” techniques are well known to the person skilled in the art.

In another specific aspect of the EHV according to the present invention the EHV is recombinant.

The term “recombinant” as used herein relates to a viral genome or nucleic acid sequence having any modifications that do not naturally occur to the corresponding viral genome or nucleic acid sequence. For instance, a viral genome or nucleic acid sequence is considered “recombinant” if it contains an insertion, deletion, inversion, relocation or a point mutation introduced artificially, e.g., by human intervention. Therefore, the viral genome or nucleic acid sequence is not associated with all or a portion of the viral genome or nucleic acid sequence with which it is associated in nature. The term “recombinant virus” encompasses genetically modified viruses. Further, a virus comprising a heterologous or an exogenous sequence such as an exogenous antigen encoding sequence (such as a Feline antigen encoding sequence) is a recombinant virus. The term recombinant virus and the term non-naturally occurring virus are used interchangeably.

In another specific aspect of the EHV according to the present invention the EHV is selected from the group consisting of EHV-1, EHV-3, EHV-4, EHV-8 und EHV-9.

To date eight different species of equid herpesviruses have been identified, five belonging to the subfamily Alphaherpesvirinae (EHV-1, EHV-3, EHV-4, EHV-8 und EHV-9) and three to the Gammaherpesvirinae.

In another specific aspect of the EHV according to the present invention the EHV is EHV-1.

The term “EHV-1” is well known to the person skilled in the art, however, a non-limiting reference sequence for EHV-1 would be for example the wild-type EHV-1 strain ab4 (Genbank accession number AY665713.1) or RacH (Hubert 1996. Journal of Veterinary Medicine, Series B, 43: 1-14.).

In another specific aspect of the EHV according to the present invention the EHV is RacH or RacH SE.

Two licensed modified live vaccines (MLV) against EHV-1 are currently available in the USA and Europe, respectively, Rhinomune◯;™ (Boehringer Ingelheim) and Prevaccinol◯;™ (MSD). Both contain the classically attenuated EHV-1 RacH strain, which was passaged 256 times in porcine epithelial cells for attenuation (Ma et al. 2013. Vet Microbiol. 167(1-2):123-34). The mechanism of attenuation has been investigated on the molecular level. Further, RacH a very safe vaccine strain as a reversion to virulence by passaging in vaccinated animals is highly unlikely, if possible at all.

### ORF 70 Insertion Sites

In another specific aspect of the EHV according to the present invention the insertion into ORF70 (US4) is characterized by a partial deletion, truncation, substitution or modification in ORF70 (US4), wherein US5 (ORF71) remains functional.

Preferably, the insertion results in a deletion of the 801 5′ basepairs of ORF70 leaving the remaining 423 bp of the 3′end intact but abolishing expression of the orf70 gene product glycoprotein G.

In another specific aspect of the EHV according to the present invention the insertion into ORF70 is characterized by the deletion of an approximately 801 bp deletion within ORF70 for the wild-type EHV-1 strain ab4 (Genbank accession number AY665713.1), whereby the deleted portion in the wild-type ab4 genome sequence is located between nucleotides 127681 and 128482 (SEQ ID NO.: 10) or a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In another specific aspect of the EHV according to the present invention the ORF70 insertion site encompasses a theoretical 801 bp deletion within ORF70 for the wild-type EHV-1 strain ab4 (Genbank accession number AY665713.1). The deleted portion is located in the wild-type ab4 (Genbank accession number AY665713.1) genome sequence between nucleotides 127681 and 128482 (SEQ ID NO.: 10).

In another specific aspect of the EHV according to the present invention the insertion into ORF70 (US4) is characterized by the deletion of an approximately 801 bp portion within ORF70 (US4) for RacH (SEQ ID NO:11) or a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In another specific aspect of the EHV according to the present invention the EHV comprises at least one flanking region selected from the group consisting of: SEQ ID NO:12, SEQ ID NO:13, SEQ ID NO:14, SEQ ID NO:15, SEQ ID NO:16, and SEQ ID NO:17 and a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In the present invention “flanking regions” direct the recombination of the expression cassette comprising the FHV antigen encoding sequence into the EHV-1 genome. These flanking regions are naturally present in EHV-1. The flanking regions are already described in WO 2018/054837 A1.

In another specific aspect of the EHV according to the present invention the EHV comprises (i) at least one left US4 flanking region selected from the group consisting of: SEQ ID NO:12, SEQ ID NO:14, and SEQ ID NO:16, and (ii) at least one right US4 flanking region selected from the group consisting of: SEQ ID NO:13, SEQ ID NO:15, and SEQ ID NO:17.

### Promoter and Regulatory Sequences

In another specific aspect of the EHV according to the present invention the Feline Herpes Virus (FHV) Antigen encoding sequence is operably linked to a promoter sequence.

In another specific aspect of the EHV according to the present invention the gD encoding sequence and/or gB encoding sequence is operably linked to a promoter sequence.

As used herein, the term “promoter” or “promoter sequence” means a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and animals such as mammals (including horses, pigs, cattle and humans), birds or insects. A promoter can be inducible, repressible, and/or constitutive. Inducible promoters initiate increased levels of transcription from DNA under their control in response to some change in culture conditions, such as a change in temperature (Ptashne, 2014. The Journal of Biological Chemistry Vol. 289, 9, 5417-5435). Examples of promoters well known to the person skilled in the art are for example SV40 large T, HCMV and MCMV immediate early gene 1, human elongation factor alpha promoter, baculovirus polyhedrin promoter.

As used herein, the term “operably linked” is used to describe the connection between regulatory elements and a nucleic acid sequence (such as a gene or antigen encoding sequence). Typically, the nucleic acid sequence is placed under the control of one or more regulatory elements, for example, without limitation, constitutive or inducible promoters, tissue-specific regulatory elements, and enhancers. A nucleic acid sequence, a gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence. Linking is accomplished by recombinant methods known in the art, e.g. by ligation at suitable restriction sites or blunt ends or by using fusion PCR methodology. Synthetic oligonucleotide linkers or adapters can be used in accordance with conventional practice if suitable restriction sites are not present.

In another specific aspect of the EHV according to the present invention the promoter sequence is selected from the group consisting of: SV40 large T, HCMV and MCMV immediate early gene 1, human elongation factor alpha promoter, baculovirus polyhedrin promoter, a promoter consisting of or comprising p430 (SEQ ID NO:18) or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment of at least 350 contiguous nucleotides, a promoter consisting of or comprising p455 (SEQ ID NO:19) or a sequence having at least 90%, at least 93%, at least 95%, at least 96%, at least 97%, at least 98%, at least 99%, at least 99.5%, at least 99.6%, at least 99.7%, at least 99.8%, at least 99.9%, 99.95%, at least 99.98% or at least 99.99% sequence identity thereto or a fragment of at least 350 contiguous nucleotides and a promoter consisting or comprising of a truncated FHV-1 gB Promoter (SEQ ID NO:24) or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment of at least 300 contiguous nucleotides.

In another specific aspect of the EHV according to the present invention the promoter sequence is a promoter consisting of or comprising p430 (SEQ ID NO:18) or a sequence having at least 95% sequence identity thereto or a fragment of at least 400 contiguous nucleotides.

In another specific aspect of the EHV according to the present invention the promoter sequence is a promoter consisting of or comprising p455 (SEQ ID NO:19) or a sequence having at least 95% sequence identity thereto or a fragment of at least 400 contiguous nucleotides.

In any of the aforementioned disclosure of the EHV said promoter sequence is a promoter consisting of or comprising truncated FHV-1 gB Promoter (SEQ ID NO:24) or a sequence having at least 95% sequence identity thereto or a fragment of at least 350 contiguous nucleotides.

In another specific aspect of the EHV according to the present invention the EHV comprises one or more further regulatory sequences such as a termination signal, a polyadenylation signal or a regulatory element like IRES.

The “termination signal” or “terminator” or “polyadenylation signal” or “polyA” or transcription termination site” or “transcription termination element” is a signal sequence which causes cleavage at a specific site at the 3′ end of the eukaryotic mRNA and post-transcriptional incorporation of a sequence of about 100-200 adenine nucleotides (polyA tail) at the cleaved 3′ end, and thus causes RNA polymerase to terminate transcription. The polyadenylation signal comprises the sequence AATAAA about 10-30 nucleotides upstream of the cleavage site and a sequence located downstream. Various polyadenylation elements are known such as tk polyA, SV40 late and early polyA, BGH polyA (described for example in U.S. Pat. No. 5,122,458) or hamster growth hormone polyA (WO2010010107).

In another specific aspect of the EHV according to the present invention the EHV comprises a polyadenylation signal.

In another specific aspect of the EHV according to the present invention the EHV comprises the polyadenylation signal BGHpA.

An “internal ribosome entry site” or “IRES” describes a sequence which functionally promotes translation initiation independent from the gene 5′ of the IRES and allows two cistrons (open reading frames) to be translated from a single transcript in a cell. In a eukaryotic cell, a polycistronic transcript having an IRES operably linked to the second or subsequent open reading frame in the transcript allows the sequential translation of that downstream open reading frame to produce the two or more polypeptides encoded by the same transcript. The IRES can be of varying length and from various sources, e.g. Encephalomyocarditis virus (EMCV), picornaviruses (e.g. Foot-and-mouth disease virus, FMDV or Polio virus (PV), or Hepatitis C virus (HCV). Various IRES sequences and their use in vector construction have been described and are well known in the art.

In another specific aspect of the EHV according to the present invention the EHV comprises an IRES element.

In another specific aspect of the EHV according to the present invention two FHV encoding sequences with an IRES element in between are inserted into one insertion site.

In another specific aspect of the EHV according to the present invention FHVgD with IRES and gB are inserted into the ORF1/3 site.

In another specific aspect of the EHV according to the present invention the EHV does not comprise a 2A peptide such as a F2A peptide.

The term “2a” or “2a peptide” means short oligopeptide sequences, described as 2a and ‘2a-like’, serve as linkers which are able to mediate a co-translational cleavage between proteins by a process defined as ribosomal-skipping. Such 2a and ‘2a-like’ sequences (from Picornaviridae and other viruses or cellular sequences) can be used to concatenate multiple gene sequences into a single gene, ensuring their co-expression within the same cell.

### Specific Construct

In another specific aspect of the EHV according to the present invention the FHV gD encoding sequence or a fragment thereof is inserted into ORF1/3 and the FHV gB encoding sequence or a fragment thereof is inserted into ORF70.

In another specific aspect of the EHV according to the present invention
i) the FHV gD encoding sequence consists of or comprises a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids is inserted into ORF1/3, and
ii) the FHV gB encoding sequence consists of or comprises a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids is inserted into ORF70.

### Immunogenic Composition or Vaccine

The present invention provides an immunogenic composition comprising an EHV as described herein.

The term “immunogenic composition” refers to a composition that comprises at least one antigen, which elicits an immunological response in the host to which the immunogenic composition is administered. Such immunological response may be a cellular and/or antibody-mediated immune response to the immunogenic composition of the invention. Preferably, the immunogenic composition induces an immune response and, more preferably, confers protective immunity against one or more of the clinical signs of a FHV infection. The host is also described as “subject”. Preferably, any of the hosts or subjects described or mentioned herein is a feline or a cat.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the invention. Preferably, the host will display either a protective immunological response or a therapeutically response.

A “protective immunological response” or “protective immunity” will be demonstrated by either a reduction or lack of clinical signs normally displayed by an infected host, a quicker recovery time and/or a lowered duration of infectivity or lowered pathogen titer in the tissues or body fluids or excretions of the infected host.

In case where the host displays a protective immunological response such that resistance to new infection will be enhanced and/or the clinical severity of the disease reduced, the immunogenic composition is described as a “vaccine”.

Further, the present invention provides a vaccine comprising an EHV as described herein.

Furthermore, the present invention provides a DIVA vaccine comprising an EHV as described herein.

The term “DIVA (differentiation between infected and vaccinated animals)” refers to a vaccine that can be used for differentiating a vaccinated animal from a naturally infected animal.

In another specific aspect of the immunogenic composition, vaccine or DIVA vaccine according to the present invention the immunogenic composition, vaccine or DIVA vaccine is bivalent.

In another specific aspect of the immunogenic composition, vaccine or DIVA vaccine according to the present invention the immunogenic composition, vaccine or DIVA vaccine is multivalent.

In another specific aspect of the immunogenic composition, vaccine or DIVA vaccine according to the present invention the immunogenic composition, vaccine or DIVA vaccine further comprises a pharmaceutically acceptable carrier.

The term “pharmaceutical-acceptable carrier” includes any and all solvents, dispersion media, coatings, stabilizing agents, diluents, preservatives, antibacterial and antifungal agents, isotonic agents, adsorption delaying agents, adjuvants, immune stimulants, and combinations thereof.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

In another specific aspect of the immunogenic composition, vaccine or DIVA vaccine according to the present invention said pharmaceutically acceptable carrier is aqua ad injectionem, cell culture media or a resuspension buffer.

In another specific aspect of the immunogenic composition, vaccine or DIVA vaccine according to the present invention said resuspension buffer is a physiological solution or phosphate buffered saline.

In another specific aspect of the immunogenic composition, vaccine or DIVA vaccine according to the present invention the immunogenic composition, vaccine or DIVA vaccine comprises 1×10⁴ to 1×10⁹ TCID₅₀, preferably between 1×10⁴ to 1×10⁸ TCID₅₀, even more preferably 1×10⁴ to 1×10⁷ TCID₅₀ of the EHV.

### Kits

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration preferably for administration to an animal, especially feline, preferably cat. Associated with such container(s) can be a notice in the form prescribed by a governmental agency regulating the manufacture, use or sale of pharmaceuticals or biological products, which notice reflects approval by the agency of manufacture, use or sale for animal administration.

Furthermore, the present invention provides a kit comprising the immunogenic composition, vaccine or DIVA vaccine as described herein.

In another specific aspect of the kit according to the present invention the kit further comprises an instruction letter for the treatment and/or prophylaxis of diseases of felines.

In another specific aspect of the kit according to the present invention the kit further comprises an instruction letter for the treatment and/or prophylaxis of FHV.

In another specific aspect of the kit according to the present invention the kit further comprises a dispenser capable of administering a vaccine to an animal or feline.

### Method of Treatment

The present invention provides a method for immunizing comprising administering to a feline an immunogenic composition, vaccine or a DIVA vaccine as described herein.

The present invention provides a method for immunizing a feline comprising administering to such feline an immunogenic composition, vaccine or a DIVA vaccine as described herein.

Preferably, immunization results in lessening of the incidence of the particular Feline Herpes Virus infection in a group or in the reduction in the severity of clinical signs caused by or associated with the particular Feline Herpes Virus infection.

Further, the immunization of felines with the immunogenic compositions as provided herewith, results in preventing infection of felines by Feline Herpes Virus infection. Even more preferably, immunization results in an effective, long-lasting, immunological-response against Feline Herpes Virus infection. It will be understood that the said period of time will last more than 2 months, preferably more than 3 months, more preferably more than 4 months, more preferably more than 5 months, more preferably more than 6 months. It is to be understood that immunization may not be effective in all animals immunized. However, the term requires that a significant portion of animals of a group are effectively immunized.

Preferably, a group of felines is envisaged in this context which normally, i.e. without immunization, would develop clinical signs normally caused by or associated with a Feline Herpes Virus infection. Whether the feline of a group are effectively immunized can be determined without further ado by the person skilled in the art. Preferably, the immunization shall be effective if clinical signs in at least 33%, at least 50%, at least 60%, at least 70%, at least 80%, at least 90%, still more preferably in at least 95% and most preferably in 100% of the animals of a given group are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, still more preferably by at least 95% and most preferably by 100% in comparison to felines that are either not immunized or immunized with an immunogenic composition that was available prior to the present invention but subsequently infected by the particular Feline Herpes Virus.

The present invention provides a method for the treatment or prophylaxis of clinical signs caused by FHV in felines comprising administering to the feline a therapeutically effective amount of an immunogenic composition, vaccine or DIVA vaccine as described herein.

The present invention provides a method for the treatment or prophylaxis of clinical signs caused by FHV in felines, in comparison to a feline of a non-immunized control group, comprising administering to such feline a therapeutically effective amount of an immunogenic composition, vaccine or DIVA vaccine as described herein.

Advantageously, the experimental data provided by the present invention disclose safety and efficacy of the immunogenic composition provided herein when administered to felines. In fact, felines vaccinated with the immunogenic composition provided herein have reduced clinical signs associated with the disease compared to non-vaccinated felines such as reduced rhinitis, ocular discharge or conjunctivitis after challenge virus infection.

Preferably, the clinical signs are reduced by at least 50%, even more preferably by at least 60%, still more preferably by at least 70%, even more preferably by at least 80%, even more preferably by at least 90%, still more preferably by at least 95% most preferably by 100% in comparison to a feline that is not treated (not immunized) but subsequently infected by the particular Feline Herpes Virus.

The term “treatment and/or prophylaxis” refers to the lessening of the incidence of the particular FHV infection in a group or the reduction in the severity of clinical signs caused by or associated with the particular FHV infection. Thus, the term “treatment and/or prophylaxis” also refers to the reduction of the number of animals in a group that become infected with the particular FHV (=lessening of the incidence of the particular FHV infection) or to the reduction of the severity of clinical signs normally associated with or caused by a FHV infection in a group of animals which animals have received an effective amount of the immunogenic composition as provided herein in comparison to a group of animals which animals have not received such immunogenic composition.

The “treatment and/or prophylaxis” generally involves the administration of an effective amount of the immunogenic composition of the present invention to an animal or group of animals in need of or that could benefit from such a treatment/prophylaxis. The term “treatment” refers to the administration of the effective amount of the immunogenic composition once the animal or at least some animals of the group is/are already infected with such FHV and wherein such animals already show some clinical signs caused by or associated with such FHV infection. The term “prophylaxis” refers to the administration to an animal prior to any infection of such animal with FHV or at least where such animal or none of the animals in a group of animals do not show any clinical signs caused by or associated with the infection by such FHV. The terms “prophylaxis” and “preventing” are used interchangeable in this application.

The term “clinical signs” as used herein refers to signs of infection of an animal from FHV. Examples for such clinical signs include but are not limited to respiratory distress, rhinitis, slight fever, depression, and reduced appetite. However, the clinical signs also include but are not limited to clinical signs that are directly observable from a live animal. Examples for clinical signs that are directly observable from a live animal include nasal and ocular discharge, conjunctivitis, lethargy, coughing, wheezing, thumping, elevated fever, weight loss, dehydration, lameness, wasting, paleness of the skin, unthriftiness and the like.

As used herein, the term “effective amount” means, in the context of a composition, an amount of an immunogenic composition capable of inducing an immune response that reduces the incidence of or lessens the severity of infection or incident of disease in an animal. Such effective amount is able to lessen the incidence of the particular FHV infection in a herd or to reduce the severity of clinical signs of the particular FHV infection. Particularly, an effective amount refers to colony forming units (CFU) per dose. Alternatively, in the context of a therapy, the term “effective amount” refers to the amount of a therapy which is sufficient to reduce or ameliorate the severity or duration of a disease or disorder, or one or more symptoms thereof, prevent the advancement of a disease or disorder, cause the regression of a disease or disorder, prevent the recurrence, development, onset, or progression of one or more symptoms associated with a disease or disorder, or enhance or improve the prophylaxis or treatment of another therapy or therapeutic agent.

The present invention provides a method for the treatment or prophylaxis of respiratory disease caused by FHV in felines comprising administering to the feline a therapeutically effective amount of an immunogenic composition, vaccine or DIVA vaccine as described herein.

The present invention provides a method for the treatment or prophylaxis of respiratory disease caused by FHV in felines, in comparison to a feline of a non-immunized control group, comprising administering to such feline a therapeutically effective amount of an immunogenic composition, vaccine or DIVA vaccine as described herein.

The term “respiratory disease” refers to infections of the respiratory system and encompasses clinical signs such as ocular or nasal discharge, conjunctivitis, rhinitis, sneezing, coughing and fever.

Preferably, the respiratory disease is reduced by at least 50%, even more preferably by at least 60%, still more preferably by at least 70%, even more preferably by at least 80%, even more preferably by at least 90%, still more preferably by at least 95% most preferably by 100% in comparison to a feline of a non-immunized control group of the same species that is subsequently infected by the particular Feline Herpes Virus.

The present invention provides a method for reducing shedding of FHV in felines comprising administering to the feline a therapeutically effective amount of an immunogenic composition, vaccine or DIVA vaccine as described herein.

The present invention provides a method for reducing shedding of FHV in felines, in comparison to a feline of a non-immunized control group, comprising administering to such feline a therapeutically effective amount of an immunogenic composition, vaccine or DIVA vaccine as described herein.

The term “shedding” refers to secretions such as nasal or ocular discharges and, further, to aerosols created by coughing or sneezing. Thus, shedding may be determined by examining the virus titer in nasal or ocular swabs or by the virus titer in the lungs. The term “shedding” further encompasses the transfer of virus to susceptible animals (i.e. sentinels). It is in the general knowledge of a person skilled in the art how to measure the viral shedding.

The term “reducing” means, that the shedding is reduced by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, even more preferably by at least 90%, still more preferably by at least 95% most preferably by 100% in comparison to subjects that are not treated (not immunized) but subsequently infected by the particular FHV.

### EP Format

The present invention provides the immunogenic composition, vaccine or DIVA vaccine as described herein for use in a method for immunizing a feline comprising administering to such feline a therapeutically effective amount of said immunogenic composition, vaccine or DIVA vaccine.

The present invention provides the immunogenic composition, vaccine or DIVA vaccine as described herein for use in a method for the treatment or prophylaxis of clinical signs caused by FHV in felines comprising administering to the feline a therapeutically effective amount of said immunogenic composition, vaccine or DIVA vaccine.

The present invention provides the immunogenic composition, vaccine or DIVA vaccine as described herein for use in a method for the treatment or prophylaxis of respiratory disease caused by FHV in felines comprising administering to the feline a therapeutically effective amount of said immunogenic composition, vaccine or DIVA vaccine.

The present invention provides the immunogenic composition, vaccine or DIVA vaccine as described herein for use in a method for reducing shedding of FHV in felines comprising administering to the feline a therapeutically effective amount of said immunogenic composition, vaccine or DIVA vaccine.

In another specific aspect of the method or use according to the present invention the feline is selected from a list consisting of: cheetah, puma, jaguar, leopard, lion, lynx, tiger, cat and domestic cat.

In another specific aspect of the method or use according to the present invention the feline is a cat or domestic cat.

However, the immunogenic composition can be administered to the feline at two or more doses, with a first dose being administered prior to the administration of a second (booster) dose.

In another specific aspect of the method or use according to the present invention the immunogenic composition, vaccine or DIVA vaccine is administered in two or more doses.

In another specific aspect of the method or use according to the present invention the immunogenic composition, vaccine or DIVA vaccine is administered in two doses.

As shown in the Examples the immunogenic composition as provided herein has been proven to be efficacious after the administration of two doses.

In addition to the first and second dose regimen, an alternate embodiment comprises further subsequent doses. For example, a third, fourth, or fifth dose could be administered in these aspects. Preferably, subsequent third, fourth, and fifth dose regimens are administered in the same amount as the first dose, with the time frame between the doses being consistent with the timing between the first and second doses mentioned below. In one aspect of the present invention the feline is a cat.

Preferably, the dose has a total volume between about 0.25 ml and 2.5 ml, more preferably between about 0.25 ml and 1.5 ml and even more preferably between about 0.5 ml and 1 mi.

In another specific aspect of the method or use according to the present invention the immunogenic composition, vaccine or DIVA vaccine is administered to the feline from an age of 4, 5 or 6 weeks onwards.

In another specific aspect of the method or use according to the present invention the immunogenic composition, vaccine or DIVA vaccine is administered to the feline from an age of 6 weeks onwards.

In another specific aspect of the method or use according to the present invention the immunogenic composition, vaccine or DIVA vaccine is administered for the first time to the feline within 4 weeks of age to 25 weeks of age.

In another specific aspect of the method or use according to the present invention the immunogenic composition, vaccine or DIVA vaccine is administered for the first time to the feline within 6 weeks of age to 20 weeks of age.

In another specific aspect of the method or use according to the present invention a first dose of the immunogenic composition, vaccine or DIVA vaccine is administered to the feline from an age of 4, 5 or 6 weeks onwards and the second dose is administered 2 to 8 weeks later.

In another specific aspect of the method or use according to the present invention a first dose of the immunogenic composition, vaccine or DIVA vaccine is administered to the feline from an age of 6 weeks onwards and the second dose is administered 2 to 8 weeks later.

In another specific aspect of the method or use according to the present invention after the second (booster) vaccination a further vaccination is done on a yearly basis (an annual booster vaccination).

The immunogenic composition, vaccine or DIVA vaccine is, preferably, administered topically or systemically. Suitable routes of administration conventionally used are oral or parenteral administration, such as intranasal, intravenous, intramuscular, intraperitoneal, subcutaneous, as well as inhalation. However, depending on the nature and mode of action of a compound, the immunogenic composition, vaccine or DIVA vaccine may be administered by other routes as well. However, most preferred the immunogenic composition, vaccine or DIVA vaccine is administered intramuscular, subcutaneous or intranasal.

In another specific aspect of the method or use according to the present invention said immunogenic composition, vaccine or DIVA vaccine is administered intramuscular, intradermal, subcutaneous, orally or nasally.

In another specific aspect of the method or use according to the present invention the immunogenic composition, vaccine or DIVA vaccine comprises 1×10⁴ to 1×10¹⁰ TCIDso of the EHV per ml.

In another specific aspect of the method or use according to the present invention the immunogenic composition, vaccine or DIVA vaccine comprises 1×10⁶ to 1×10⁹ TCIDso of the EHV per ml.

In another specific aspect of the method or use according to the present invention one dose of the immunogenic composition, vaccine or DIVA vaccine comprises 1×10⁴ to 1×10¹⁰ TCID₅₀ of the EHV.

In another specific aspect of the method or use according to the present invention one dose of the immunogenic composition, vaccine or DIVA vaccine comprises 1×10⁶ to 1×10⁹ TCID₅₀ of the EHV.

In another specific aspect of the method or use according to the present invention said method results in an improvement in an efficacy parameter selected from the group consisting of: a reduction in weight loss, a lower virus load in lungs, a reduction in viremia, a reduction in lung lesions, reduced respiratory and alveolar epithelial lesions, a reduced ocular discharge, a reduced conjunctivitis, a reduced rhinitis, a reduced and/or shortened shedding of virus, a reduced rectal temperature, reduced clinical symptoms (in particular respiratory symptoms), increased induction of (neutralizing) anti-FHV virus antibodies, increased stimulation of T-cells against FHV, increased stimulation of B-cells against FHV virus, and a reduction of proinflammatory cytokines, e.g. IL1, in lungs, or combinations thereof, in comparison to a feline of a non-immunized control group of the same species.

The term “virus load” is well known to the person skilled in that art. The term virus load is interchangeable used with the term “viral titer” herein. The virus load or virus titer is a measure of the severity of an active viral infection, and can be determined by methods known to the person skilled in the art. The determination can be based on the detection of viral proteins such as by antibody binding to the viral proteins and further detection or, alternatively, by detection of viral nucleic acids by amplification methods such as RT-PCR. Monitoring of virion associated viral RNA in plasma by nucleic acid amplification methods is a widely used parameter to assess the status and progression of retroviral disease, and to evaluate the effectiveness of prophylactic and therapeutic interventions. Exemplary, the virus load or virus titer can be calculated by estimating the live amount of virus in an involved body fluid such as a number of RNA copies per milliliter of blood plasma. Preferably, the term “virus load” or “virus titer” is a measure of infectious units per volume of a virus preparation. Viral titer is an endpoint in a biological procedure and is defined as the dilution at which a certain proportion of tests carried out in parallel show an effect (Reed and Muench, 1938). Specifically the tissue culture infectious dose fifty per milliliter (TCID50/ml) gives the dilution of a virus preparation at which 50% of a number of cell cultures inoculated in parallel with that dilution are infected.

In another specific aspect of the method or use according to the present invention the immunogenic composition, vaccine or a DIVA vaccine protects against a homologous and/or heterologous challenge with a FHV.

The experimental part shows that the EHV vaccine carrying FHV antigens (vaccine strain C-27) protects against a heterologous challenge (SGE strain, named FVR 96-13).

### Independent Picture EHV's

The present invention provides an EHV (Equine Herpesvirus) comprising a FHV gD encoding sequence or a fragment thereof inserted into ORF1/3 and a FHV gB encoding sequence or a fragment thereof inserted into ORF70.

The present invention provides an EHV (Equine Herpesvirus) comprising:
i) a FHV gD encoding sequence consisting of or comprising a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 200, at least 300 or at least 350 consecutive amino acids, inserted into ORF1/3, and
ii) a FHV gB encoding sequence consisting of or comprising a nucleic acid sequence encoding an amino acid sequence as set forth in SEQ ID NO:5 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 150, at least 200, at least 300, at least 400, at least 500, at least 600, at least 700 or at least 800 consecutive amino acids, inserted into ORF70.

The present invention provides an EHV (Equine Herpesvirus) comprising a FHV gD encoding sequence consisting of or comprising a nucleic acid sequence encoding an amino acid sequence as set forth in SEQ ID NO:1 or a sequence having at least 95% sequence identity thereto inserted into ORF1/3 and a FHV gB encoding sequence consisting of or comprising a nucleic acid sequence encoding an amino acid sequence as set forth in SEQ ID NO:2 or a sequence having at least 95% sequence identity thereto inserted into ORF70.

The present invention provides an immunogenic composition, vaccine or DIVA vaccine comprising an EHV (Equine Herpesvirus) comprising:
i) a FHV gD encoding sequence consisting of or comprising a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 200, at least 300 or at least 350 consecutive amino acids, inserted into ORF1/3, and
ii) a FHV gB encoding sequence consisting of or comprising a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 150, at least 200, at least 300, at least 400, at least 500, at least 600, at least 700 or at least 800 consecutive amino acids, inserted into ORF70.

The present invention further provides an EHV, immunogenic composition, vaccine or DIVA vaccine as described herein for therapeutic use.

The present invention further provides an EHV, immunogenic composition, vaccine or DIVA vaccine as described herein for use as an immunogen or vaccine.

The present invention further provides an EHV, immunogenic composition, vaccine or DIVA vaccine as described herein for use as a medicament.

The present invention further provides an EHV, immunogenic composition, vaccine or DIVA vaccine as described herein for the manufacture of a medicament.

The present invention further provides the use of an EHV, immunogenic composition, vaccine or DIVA vaccine as described herein for the treatment and/or prophylaxis of FHV infections in felines.

### Method of Production

The present invention further provides a method of preparing an EHV (Equine Herpesvirus) comprising a Feline Herpes Virus (FHV) Antigen encoding sequence inserted into ORF70 (US4) and/or ORF1/3 comprising:
i) providing an EHV; and
ii) providing a FHV Antigen encoding sequence; and
iii) inserting said FHV Antigen encoding sequence from step ii) into the ORF70 (US4) and/or ORF1/3 insertion site of the EHV of step i); and
iv) obtaining said EHV comprising a FHV Antigen encoding sequence inserted into ORF70 (US4) and/or ORF1/3.

The present invention further provides a method of preparing an EHV (Equine Herpesvirus) comprising a Feline Herpes Virus (FHV) Antigen encoding sequence inserted into ORF70 (US4) and/or ORF1/3 comprising:
i) providing an EHV; and
ii) providing a FHV gD, gB or gC encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids; and
iii) inserting said FHV gD, gB or gC encoding sequence from step ii) into the ORF70 (US4) and/or ORF1/3 insertion site of the EHV of step i); and
iv) obtaining said EHV comprising FHV gD, gB or gC encoding sequence inserted into ORF70 (US4) and/or ORF1/3.

The term “obtaining” comprises the harvest, isolation, purification and/or formulation (e.g. finishing, inactivation and/or blending) of said EHV with the FHV Antigen encoding sequence.

The term “harvest” refers to collecting or recovering said EHV with the FHV Antigen encoding sequence from the transfected or infected cell, bacteria or cell line. Any conventional method known in the art can be used, e.g. any separation method. Well known methods in the art comprise centrifugation or filtration, such as using a semi-permeable membrane having a certain pore size.

The term “isolation” comprises an isolation step of said EHV with the FHV Antigen encoding sequence. Methods for the isolation from the transfected or infected cell, bacteria or cell line are known to a person skilled in the art. Those methods comprise physical and/or chemical methods, including but are not limited to freeze thaw cycles, treatment with ultrasound and the alike.

Methods for the “purification” of said EHV with the FHV Antigen encoding sequence from the isolate are known to a person skilled in the art, for example by those methods described in Protein purification methods—a practical approach (E.L.V. Harris and S. Angel, eds., IRL Press at Oxford University Press). Those methods include, but are not limited to, separation by centrifugation and/or filtration, precipitation, size exclusion (gel filtration) chromatography, affinity chromatography, metal chelate chromatography, ion-exchange chromatography covalent chromatography, hydrophobic interaction chromatography, and the alike. The vector can be obtained in a purified pure form, or free or substantially free of other cellular materials or culture medium etc. After said isolation and/or purification the antigen exhibits a purity of at least 80%, preferably 80%-90%, more preferably 90%-97%, most preferred more than 97% up to an absolute pure form without any contamination.

According to a further aspect, “obtaining” as used herein may also include further finishing steps as part of the final formulation process, like the addition of buffer, inactivation, neutralization steps and the alike.

In another specific aspect of the method of preparing an EHV comprising a FHV Antigen encoding sequence inserted into ORF70 (US4) and/or ORF1/3 according to the present invention the EHV is attenuated.

In another specific aspect of the method of preparing an EHV comprising a FHV Antigen encoding sequence inserted into ORF70 (US4) and/or ORF1/3 according to the present invention the EHV is RacH or RacH SE.

### Diva

The DIVA vaccine facilitates fast and effective administration and allows discrimination between animals infected with the field virus (disease-associated) and vaccinated animals.

The immunogenic composition, vaccine or DIVA vaccine of the present invention does not comprise any FHV gG antigen encoding sequence.

In contrast, after infection of animals with wild-type Feline Herpes Virus or vaccinated with a modified live vaccine or vaccinated with an inactivated whole virus vaccine, the infected/vaccinated animals produce/have specific antibodies against FHV gG. However, in animals vaccinated with the immunogenic composition according to the present invention such specific antibodies against FHV gG cannot be detected.

The immunogenic composition, vaccine or DIVA vaccine of the present invention comprise an EHV and EHV gD and EHV gC encoding sequences, respectively. For that reason, in animals vaccinated with the immunogenic composition according to the present invention specific antibodies against EHV such as antibodies against EHV gD and/or EHV gC can be detected.

In contrast, after infection of animals with wild-type Feline Herpes Virus or vaccinated with a modified live vaccine or vaccinated with an inactivated whole virus vaccine, the animals will be negative for EHV specific antibodies such as antibodies against EHV gD and/or EHV gC. In contrast, in animals vaccinated with the immunogenic composition according to the present invention such specific antibodies against EHV gD and gC can be detected.

By exemplary immuno tests and/or genomic analytical tests the animals vaccinated with the immunogenic composition of the present invention can be differentiated from animals that were infected with the wildtype Feline Herpes Virus or vaccinated with a modified live vaccine or vaccinated with an inactivated whole virus vaccine a) in that animals vaccinated with the immunogenic composition of the present invention do not have any specific antibodies against FHV gD and any FHV gD encoding sequence, respectively, or b) in that animals vaccinated with the immunogenic composition of the present invention have specific antibodies against EHV gD and/or EHV gC and EHV gD and/or EHV gC encoding sequence, respectively.

The term “immuno tests” and “genomic analytical tests” is the basis for differentiating animals vaccinated with the immunogenic composition according to the present invention and animals infected with FHV.

Examples of immuno tests include any enzyme-immunological or immunochemical detection method such as ELISA (enzyme linked immunosorbent assay), EIA (enzyme immunoassay), RIA (radioimmunoassay), sandwich enzyme immune tests, fluorescent antibody test (FAT) electrochemiluminescence sandwich immunoassays (ECLIA), dissociation-enhanced lanthanide fluoro immuno assay (DELFIA) or solid phase immune tests, immunofluorescent test (IFT), immunohistological staining, Western blot analysis or any other suitable method available to technicians skilled in the art. Depending upon the assay used, the antigens or the antibodies can be labeled by an enzyme, a fluorophore or a radioisotope. See, e.g., Coligan et al. Current Protocols in Immunology, John Wiley & Sons Inc., New York, N.Y. (1994); and Frye et al., Oncogen 4: 1153-1157, 1987.

The term “genomic analytical test” refers to a genomic analytical method based upon the polymerase chain reaction (PCR), reverse transcription polymerase chain reaction (RT-PCR), real-time PCR (r-PCR) or real time reverse transcription PCR (rRT-PCR), Templex-PCR, nucleic-acid sequence based amplification (NASBA), and isothermal amplification methods using polymerases and specific oligonucleotides as primers. The aforementioned amplification methods are well known in the art.

The present invention provides a method of differentiating felines infected with FHV from felines vaccinated with the immunogenic composition, vaccine or the DIVA vaccine as described herein, comprising
a) obtaining a sample from a feline, and
b) analyzing said sample in an immuno test, cell culture based assay and/or genomic analytical test.

Advantageously, the experimental part of the present application already describes how such analyzing of the samples could be done. Example 3 and 4 describes an IFA (Immunofluorescence Assay) for measuring the expression of gD and gB (immuno test). In Examples 4 and 5 the FHV SN and EHV SN assay readouts are based on a cell culture based assay, a CPE (Cytopathic effect or cytopathogenic effect) assay.

Further, Maes 2012 (ISRN Veterinary Science. (2-3):495830) reviews different methods how to diagnose FHV infection. The most common laboratory diagnostic methods to demonstrate the presence of FHV or viral components in tissue homogenates or swabs include the direct fluorescent antibody (FA) test, virus isolation (VI), and PCR. Fluorescent antibody testing is performed on conjunctival or corneal tissue. Virus isolation (VI) or PCR is using oronasal and conjunctival swab extracts as the samples. VI detects infectious virus and has been the laboratory diagnostic gold standard. However, multiple PCR assays have been described for use in the detection of FHV as well.

The term “sample” refers to a sample of a body fluid, to a sample of separated cells or to a sample from a tissue or an organ. Samples of body fluids can be obtained by well-known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. However, the term “sample” also covers oropharyngeal, conjunctival or nasal swabs.

The term “obtained” may comprise an isolation and/or purification step known to the person skilled in the art, preferably using precipitation, columns ect.

In one specific aspect of the method of differentiating according to the present invention the immuno test comprises testing whether the sample comprises antibodies specifically recognizing FHV gG.

In another specific aspect of the method of differentiating according to the present invention the feline is infected with FHV if antibodies specifically recognizing FHV gG have been detected.

In another specific aspect of the method of differentiating according to the present invention the genomic analytical test comprises testing whether the sample comprises a FHV gG encoding sequence.

In another specific aspect of the method of differentiating according to the present invention the feline is infected with FHV if a FHV gG encoding sequence has been detected.

In another specific aspect of the method of differentiating according to the present invention the immuno test comprises testing whether the sample comprises antibodies specifically recognizing EHV gD and/or EHV gC.

In another specific aspect of the method of differentiating according to the present invention the feline is vaccinated with the immunogenic composition, vaccine or DIVA vaccine as described herein if antibodies specifically recognizing the EHV gD and/or EHV gC have been detected.

In another specific aspect of the method of differentiating according to the present invention the genomic analytical test comprises testing whether the sample comprises a EHV gD and/or EHV gC encoding sequence.

In another specific aspect of the method of differentiating according to the present invention the feline is vaccinated with the immunogenic composition, vaccine or DIVA vaccine as described herein if a EHV gD and/or EHV gC encoding sequence has been detected.

In another specific aspect of the method of differentiating according to the present invention the immuno test is an EIA (enzyme immunoassay) or ELISA (enzyme linked immunosorbent assay), or, wherein the genomic analytical test is a PCR (polymerase chain reaction), RT-PCR (reverse transcriptase polymerase chain reaction) or real time PCR (polymerase chain reaction).

In another specific aspect of the method of differentiating according to the present invention the feline is a cat.

In another specific aspect of the method of differentiating according to the present invention the sample is a serum sample.

In another specific aspect of the method of differentiating according to the present invention the ELISA is an indirect ELISA, Sandwich ELISA, a competitive ELISA or blocking ELISA.

Thus, a test could e.g. comprise wells with a FHV gG (or alternatively EHV gD and/or EHV gC) epitope cross-linked to micro-well assay plates. Said cross-linking preferably is performed through an anchor protein such as, for example, poly-L-lysine. Expression systems for obtaining FHV gG (or alternatively EHV gD and/or EHV gC) epitopes are well known to the person skilled in the art. Alternatively, said FHV gG (or alternatively EHV gD and/or EHV gC) epitopes could be chemically synthesized.

Animals only vaccinated with the vaccine according to the present invention have not raised antibodies against the FHV gG epitope. However, such animals have raised antibodies against EHV gD and/or EHV gC. As a consequence, no antibodies bind to a well coated with the FHV gG epitope. However, such vaccinated animals have raised antibodies against EHV gD and/or EHV gC. Therefore, if a well has been coated with an EHV gD and/or EHV gC epitope antibodies would bind to such epitope and would give a positive detection signal.

However, the different ELISA techniques are well known to the person skilled in the art. ELlSA's have been described exemplary by Wensvoort G. et al., 1988 (Vet. Microbiol. 17(2): 129-140), by Robiolo B. et al., 2010 (J. Virol. Methods. 166(1-2): 21-27) and by Colijn, E. O. et al., 1997 (Vet. Microbiology 59: 15-25).

Preferably, the test for differentiating an animal that is infected with FHV or vaccinated with a modified live vaccine or vaccinated with an inactivated whole virus vaccine and such that are only vaccinated with the vaccine of the present invention is provided by RNA isolation of cells from a sample as defined above and reverse transcriptase followed by amplification of the cDNA. Using specific primers for FHV gG a PCR can be performed. In such a case the feline is infected with FHV if there is a positive PCR signal. However, if no FHV gG specific sequence can be amplified, the feline is not infected with FHV, but may have been vaccinated with the vaccine of the present invention. Using specific primers for EHV gD and/or EHV gC a PCR can be performed. In such a case the feline has been vaccinated with the vaccine of the present invention if there is a positive PCR signal.

In another specific aspect of the method of differentiating according to the present invention the genomic analytical test is a PCR (polymerase chain reaction), RT-PCR (reverse transcriptase polymerase chain reaction) or real time PCR (polymerase chain reaction).

In another specific aspect of the method of differentiating according to the present invention the cell culture based assay is a CPE (Cytopathic effect or cytopathogenic effect) assay.

However, “cell culture based assays” or “CPE assay” techniques are well known to the person skilled in the art.

### DISCLOSURE

The disclosure further comprises:
An EHV (Equine Herpesvirus) comprising a Feline Herpes Virus (FHV) Antigen encoding sequence inserted into ORF70 (US4) and/or ORF1/3.

The disclosure further comprises:
An EHV (Equine Herpesvirus) comprising one single Feline Herpes Virus (FHV) Antigen encoding sequence inserted into ORF70 (US4) and/or one single Feline Herpes Virus (FHV) Antigen encoding sequence inserted into ORF1/3.

The disclosure further comprises:
An EHV (Equine Herpesvirus) comprising a Feline Herpes Virus (FHV) Antigen encoding sequence encoding equal or less than 1400 amino acids inserted into ORF70 (US4) and/or ORF1/3.

In any of the aforementioned disclosure of the EHV said Feline Herpes Virus (FHV) Antigen encoding sequence is inserted into ORF70 (US4).

In any of the aforementioned disclosure of the EHV said Feline Herpes Virus (FHV) Antigen encoding sequence is inserted into ORF1/3.

In any of the aforementioned disclosure of the EHV said Feline Herpes Virus (FHV) Antigen encoding sequence is inserted into ORF70 (US4) and ORF1/3.

In any of the aforementioned disclosure of the EHV said FHV Antigen encoding sequence encodes equal or less than 1400 amino acids.

In any of the aforementioned disclosure of the EHV said FHV Antigen encoding sequence encodes equal or less than 1200 amino acids.

In any of the aforementioned disclosure of the EHV said FHV Antigen encoding sequence encodes equal or less than 1000 amino acids.

In any of the aforementioned disclosure of the EHV said FHV Antigen encoding sequence encodes between 200 and 1200 amino acids.

In any of the aforementioned disclosure of the EHV said FHV Antigen encoding sequence encodes between 200 and 1000 amino acids.

In any of the aforementioned disclosure of the EHV said FHV Antigen encoding sequence comprises equal or less than 4200 nucleic acids.

In any of the aforementioned disclosure of the EHV said FHV Antigen encoding sequence comprises equal or less than 3600 nucleic acids.

In any of the aforementioned disclosure of the EHV said FHV Antigen encoding sequence comprises equal or less than 3000 nucleic acids.

In any of the aforementioned disclosure of the EHV said FHV Antigen encoding sequence encodes between 600 and 3600 nucleic acids.

In any of the aforementioned disclosure of the EHV said FHV Antigen encoding sequence encodes between 600 and 3000 nucleic acids.

In any of the aforementioned disclosure of the EHV said FHV Antigen encoding sequence is one FHV Antigen encoding sequence.

In any of the aforementioned disclosure of the EHV said FHV Antigen encoding sequence is one single FHV Antigen encoding sequence.

In any of the aforementioned disclosure of the EHV said FHV Antigen encoding sequence is not two or more FHV Antigen encoding sequences.

In any of the aforementioned disclosure of the EHV said FHV Antigen encoding sequence is a glycoprotein encoding sequence or a fragment thereof.

In any of the aforementioned disclosure of the EHV said FHV Antigen encoding sequence is a FHV gD (glycoprotein D) encoding sequence or a fragment thereof and/or a FHV gB (glycoprotein B) encoding sequence or a fragment thereof and/or a FHV gC (glycoprotein C) encoding sequence or a fragment thereof.

In any of the aforementioned disclosure of the EHV said FHV Antigen encoding sequence is a FHV gD encoding sequence or a fragment thereof.

In any of the aforementioned disclosure of the EHV said FHV Antigen encoding sequence is a FHV gB encoding sequence or a fragment thereof.

In any of the aforementioned disclosure of the EHV said FHV Antigen encoding sequence is a FHV gC encoding sequence or a fragment thereof.

In any of the aforementioned disclosure of the EHV said fragment of the glycoprotein encoding sequence encodes at least 100, at least 150, at least 200, at least 300, at least 350, at least 400, at least 500, at least 600, at least 700 or at least 800 amino acids.

In any of the aforementioned disclosure of the EHV said fragment of the glycoprotein encoding sequence comprises at least 300, at least 450, at least 600, at least 900, at least 1050, at least 1200, at least 1500, at least 1800, at least 2100 or at least 2400 nucleic acids.

In any of the aforementioned disclosure of the EHV said fragment of the glycoprotein encoding sequence encodes at least 100, at least 200, at least 300 or at least 350 amino acids.

In any of the aforementioned disclosure of the EHV said fragment of the glycoprotein encoding sequence comprises at least 300, at least 600, at least 900 or at least 1050 nucleic acids.

In any of the aforementioned disclosure of the EHV said fragment of the FHV gD encoding sequence encodes of at least 100, at least 200, at least 250, at least 300 or at least 350 amino acids.

In any of the aforementioned disclosure of the EHV said fragment of the FHV gD encoding sequence comprises at least 300, 600, 750, 900 or 1050 nucleic acids.

In any of the aforementioned disclosure of the EHV said fragment of the FHV gB encoding sequence encodes at least 100, at least 150, at least 200, at least 300, at least 400, at least 500, at least 600, at least 700 or at least 800 amino acids.

In any of the aforementioned disclosure of the EHV said fragment of the FHV gB encoding sequence comprises at least 300, at least 450, at least 600, at least 900, at least 1200, at least 1500, at least 1800, at least 2100 or at least 2400 nucleic acids.

In any of the aforementioned disclosure of the EHV said fragment of the FHV gC encoding sequence encodes at least 100, at least 150, at least 200, at least 300, at least 400 or at least 500 amino acids.

In any of the aforementioned disclosure of the EHV said fragment of the FHV gC encoding sequence comprises at least 300, at least 450, at least 600, at least 900, at least 1200 or at least 1500 nucleic acids.

In any of the aforementioned disclosure of the EHV said FHV Antigen encoding sequence is a FHV gD encoding sequence and/or a FHV gB encoding sequence and/or a FHV gC encoding sequence based on wild-type feline herpesvirus strain C-27 (ACCN: FJ478159).

In any of the aforementioned disclosure of the EHV said FHV Antigen encoding sequence is a FHV gD encoding sequence consisting of or comprising a nucleic acid sequence encoding the amino acid sequence as shown in AAB30980.1 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In any of the aforementioned disclosure of the EHV said FHV Antigen encoding sequence is a FHV gB encoding sequence consisting of or comprising a nucleic acid sequence encoding the amino acid sequence as shown in AAB28559.3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids.

In any of the aforementioned disclosure of the EHV said FHV Antigen encoding sequence is a glycoprotein encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In any of the aforementioned disclosure of the EHV said FHV Antigen encoding sequence is a gD, gB or gC encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In any of the aforementioned disclosure of the EHV said FHV Antigen encoding sequence is a glycoprotein consisting of or comprising a nucleic acid sequence as set forth in SEQ ID NO:4, SEQ ID NO:5, SEQ ID NO:6, SEQ ID NO:7, SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In any of the aforementioned disclosure of the EHV said FHV Antigen encoding sequence is a gD, gB or gC consisting of or comprising a nucleic acid sequence as set forth in SEQ ID NO:4, SEQ ID NO:5, SEQ ID NO:6, SEQ ID NO:7, SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In any of the aforementioned disclosure of the EHV said gD encoding sequence consists of or comprises a nucleic acid sequence encoding an amino acid sequence as set forth in SEQ ID NO:1 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

In any of the aforementioned disclosure of the EHV said gD encoding sequence consists of or comprises a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said gB encoding sequence consists of or comprises a nucleic acid sequence encoding an amino acid sequence as set forth in SEQ ID NO:2 or a sequence having at least 70%, at least 75%, at least 80%, at least 85%, at least 90%, at least 93%, at least 95%, at least 96%, at least 97%, at least 98%, at least 99%, 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids.

In any of the aforementioned disclosure of the EHV said gB encoding sequence consists of or comprises a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said gC encoding sequence consists of or comprises a nucleic acid sequence encoding an amino acid sequence as set forth in SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 150, at least 200, at least 300, at least 400 or at least 500 consecutive amino acids.

In any of the aforementioned disclosure of the EHV said gC encoding sequence consists of or comprises a nucleic acid sequence encoding an amino acid sequence as set forth in SEQ ID NO:3 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said gD encoding sequence consists of or comprises a nucleic acid sequence as set forth in SEQ ID NO:4 or SEQ ID NO:5 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

In any of the aforementioned disclosure of the EHV said gD encoding sequence consists of or comprises a nucleic acid sequence as set forth in SEQ ID NO:4 or SEQ ID NO:5 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said gB encoding sequence consists of or comprises a nucleic acid sequence as set forth in SEQ ID NO:6 or SEQ ID NO:7 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 450, at least 600, at least 900, at least 1200, at least 1500, at least 1800, at least 2100 or at least 2400 consecutive nucleic acids.

In any of the aforementioned disclosure of the EHV said gB encoding sequence consists of or comprises a nucleic acid sequence as set forth in SEQ ID NO:6 or SEQ ID NO:7 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said gC encoding sequence consists of or comprises a nucleic acid sequence as set forth in SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 450, at least 600, at least 900, at least 1200 or at least 1500 consecutive nucleic acids.

In any of the aforementioned disclosure of the EHV said gC encoding sequence consists of or comprises a nucleic acid sequence as set forth in SEQ ID NO:8 or SEQ ID NO:9 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto.

In any of the aforementioned disclosure of the EHV said EHV is a vector.

In any of the aforementioned disclosure of the EHV said EHV is an EHV-vector.

In any of the aforementioned disclosure of the EHV said EHV is attenuated.

In any of the aforementioned disclosure of the EHV said EHV is genetically engineered.

In any of the aforementioned disclosure of the EHV said EHV is recombinant.

In any of the aforementioned disclosure of the EHV said EHV is selected from the group consisting of EHV-1, EHV-3, EHV-4, EHV-8 und EHV-9.

In any of the aforementioned disclosure of the EHV said EHV is EHV-1.

In any of the aforementioned disclosure of the EHV said EHV is RacH or RacH SE.

In any of the aforementioned disclosure of the EHV said insertion into ORF70 (US4) is characterized by a partial deletion, truncation, substitution or modification in ORF70 (US4), wherein US5 (ORF71) remains functional.

In any of the aforementioned disclosure of the EHV said insertion into ORF70 is characterized by the deletion of an approximately 801 bp deletion within ORF70 for the wild-type EHV-1 strain ab4 (Genbank accession number AY665713.1), whereby the deleted portion in the wild-type ab4 genome sequence is located between nucleotides 127681 and 128482 (SEQ ID NO.: 10) or a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In any of the aforementioned disclosure of the EHV said insertion into ORF70 (US4) is characterized by the deletion of an approximately 801 bp portion within ORF70 (US4) for RacH (SEQ ID NO:11) or a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In any of the aforementioned disclosure of the EHV said EHV comprises at least one flanking region selected from the group consisting of: SEQ ID NO:12, SEQ ID NO:13, SEQ ID NO:14, SEQ ID NO:15, SEQ ID NO:16, and SEQ ID NO:17 and a sequence having at least 70%, at least 80%, at least 85%, at least 90%, at least 91%, at least 92%, at least 93%, at least 94%, at least 95%, at least 96%, at least 97%, at least 98%, at least 99% sequence identity thereto.

In any of the aforementioned disclosure of the EHV said EHV comprises (i) at least one left US4 flanking region selected from the group consisting of: SEQ ID NO:12, SEQ ID NO:14, and SEQ ID NO: 16, and (ii) at least one right US4 flanking region selected from the group consisting of: SEQ ID NO:13, SEQ ID NO:15, and SEQ ID NO:17.

In any of the aforementioned disclosure of the EHV said Feline Herpes Virus (FHV) Antigen encoding sequence is operably linked to a promoter sequence.

In any of the aforementioned disclosure of the EHV said gD encoding sequence and/or gB encoding sequence is operably linked to a promoter sequence.

In any of the aforementioned disclosure of the EHV said promoter sequence is selected from the group consisting of: SV40 large T, HCMV and MCMV immediate early gene 1, human elongation factor alpha promoter, baculovirus polyhedrin promoter, a promoter consisting or comprising of p430 (SEQ ID NO:18) or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment of at least 350 contiguous nucleotides, a promoter consisting or comprising of p455 (SEQ ID NO:19) or a sequence having at least 90%, at least 93%, at least 95%, at least 96%, at least 97%, at least 98%, at least 99%, at least 99.5%, at least 99.6%, at least 99.7%, at least 99.8%, at least 99.9%, 99.95%, at least 99.98% or at least 99.99% sequence identity thereto or a fragment of at least 350 contiguous nucleotides and a promoter consisting or comprising of a truncated FHV-1 gB Promoter (SEQ ID NO:24) or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment of at least 300 contiguous nucleotides.

In any of the aforementioned disclosure of the EHV said promoter sequence is a promoter consisting of or comprising p430 (SEQ ID NO:18) or a sequence having at least 95% sequence identity thereto or a fragment of at least 400 contiguous nucleotides.

In any of the aforementioned disclosure of the EHV said promoter sequence is a promoter consisting of or comprising p455 (SEQ ID NO:19) or a sequence having at least 95% sequence identity thereto or a fragment of at least 400 contiguous nucleotides.

In any of the aforementioned disclosure of the EHV said promoter sequence is a promoter consisting of or comprising truncated FHV-1 gB Promoter (SEQ ID NO:24) or a sequence having at least 95% sequence identity thereto or a fragment of at least 350 contiguous nucleotides.

In any of the aforementioned disclosure of the EHV said EHV comprises one or more further regulatory sequences such as a termination signal, a polyadenylation signal or a regulatory element like IRES.

In any of the aforementioned disclosure of the EHV said EHV comprises a polyadenylation signal.

In any of the aforementioned disclosure of the EHV said EHV comprises the polyadenylation signal BGHpA.

In any of the aforementioned disclosure of the EHV said EHV comprises an IRES element.

In any of the aforementioned disclosure of the EHV two FHV encoding sequences with an IRES element in between are inserted into one insertion site.

In any of the aforementioned disclosure of the EHV FHVgD with IRES and gB are inserted into the ORF1/3 site.

In any of the aforementioned disclosure of the EHV said EHV does not comprise a 2A peptide such as a F2A peptide.

In any of the aforementioned disclosure of the EHV said FHV gD encoding sequence or a fragment thereof is inserted into ORF1/3 and the FHV gB encoding sequence or a fragment thereof is inserted into ORF70.

In any of the aforementioned disclosure of the EHV:
i) the FHV gD encoding sequence consists of or comprises a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids is inserted into ORF1/3, and
ii) the FHV gB encoding sequence consists of or comprises a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, 99.9%, at least 99.95%, at least 99.98% or at least 99.99% sequence identity thereto, or a fragment thereof having at least 100, at least 150, at least 200, at least 300, at least 400, at least 500, at least 600, at least 700 or at least 800 consecutive amino acids is inserted into ORF70.

The disclosure further comprises:
An immunogenic composition comprising the disclosed EHV.

The disclosure further comprises:
A vaccine comprising the disclosed EHV.

The disclosure further comprises:
A DIVA vaccine comprising the disclosed EHV.

In any of the aforementioned disclosure of the immunogenic composition, vaccine or DIVA vaccine said immunogenic composition, vaccine or DIVA vaccine is bivalent.

In any of the aforementioned disclosure of the immunogenic composition, vaccine or DIVA vaccine said immunogenic composition, vaccine or DIVA vaccine is multivalent.

In any of the aforementioned disclosure of the immunogenic composition, vaccine or DIVA vaccine said immunogenic composition, vaccine or DIVA vaccine further comprises a pharmaceutically acceptable carrier.

In any of the aforementioned disclosure of the immunogenic composition, vaccine or DIVA vaccine said pharmaceutically acceptable carrier is aqua ad injectionem, cell culture media or a resuspension buffer.

In any of the aforementioned disclosure of the immunogenic composition, vaccine or DIVA vaccine said resuspension buffer is a physiological solution or phosphate buffered saline.

In any of the aforementioned disclosure of the immunogenic composition, vaccine or DIVA vaccine said immunogenic composition, vaccine or DIVA vaccine comprises 1×10⁴ to 1×10⁹ TCID₅₀, preferably between 1×10⁴ to 1×10⁸ TCID₅₀, even more preferably 1×10⁴ to 1×10⁷ TCID₅₀ of the EHV.

The disclosure further comprises:
A kit comprising the disclosed immunogenic composition, vaccine or DIVA vaccine.

In any of the aforementioned disclosure of the kit said kit further comprises an instruction letter for the treatment and/or prophylaxis of diseases of felines.

In any of the aforementioned disclosure of the kit said kit further comprises an instruction letter for the treatment and/or prophylaxis of FHV.

In any of the aforementioned disclosure of the kit said kit further comprises a dispenser capable of administering a vaccine to an animal or feline.

The disclosure further comprises:
A method for immunizing comprising administering to a feline the disclosed immunogenic composition, vaccine or a DIVA vaccine.

The disclosure further comprises:
A method for immunizing a feline comprising administering to such feline the disclosed immunogenic composition, vaccine or a DIVA vaccine.

The disclosure further comprises:
A method for the treatment or prophylaxis of clinical signs caused by FHV in felines comprising administering to the feline a therapeutically effective amount of the disclosed immunogenic composition, vaccine or DIVA vaccine.

The disclosure further comprises:
A method for the treatment or prophylaxis of clinical signs caused by FHV in felines, in comparison to a feline of a non-immunized control group, comprising administering to such feline a therapeutically effective amount of the disclosed immunogenic composition, vaccine or DIVA vaccine.

The disclosure further comprises:
A method for the treatment or prophylaxis of respiratory disease caused by FHV in felines comprising administering to the feline a therapeutically effective amount of the disclosed immunogenic composition, vaccine or DIVA vaccine.

The disclosure further comprises:
A method for the treatment or prophylaxis of respiratory disease caused by FHV in felines, in comparison to a feline of a non-immunized control group, comprising administering to such feline a therapeutically effective amount of the disclosed immunogenic composition, vaccine or DIVA vaccine.

The disclosure further comprises:
A method for reducing shedding of FHV in felines comprising administering to the feline a therapeutically effective amount of the disclosed immunogenic composition, vaccine or DIVA vaccine.

The disclosure further comprises:
A method for reducing shedding of FHV in felines, in comparison to a feline of a non-immunized control group, comprising administering to such feline a therapeutically effective amount of the disclosed immunogenic composition, vaccine or DIVA vaccine.

The disclosure further comprises:
The disclosed immunogenic composition, vaccine or DIVA vaccine for use in a method for immunizing a feline comprising administering to such feline a therapeutically effective amount of said immunogenic composition, vaccine or DIVA vaccine.

The disclosure further comprises:
The disclosed immunogenic composition, vaccine or DIVA vaccine for use in a method for the treatment or prophylaxis of clinical signs caused by FHV in felines comprising administering to the feline a therapeutically effective amount of said immunogenic composition, vaccine or DIVA vaccine.

The disclosure further comprises:
The disclosed immunogenic composition, vaccine or DIVA vaccine for use in a method for the treatment or prophylaxis of respiratory disease caused by FHV in felines comprising administering to the feline a therapeutically effective amount of said immunogenic composition, vaccine or DIVA vaccine.

The disclosure further comprises:
The disclosed immunogenic composition, vaccine or DIVA vaccine for use in a method for reducing shedding of FHV in felines comprising administering to the feline a therapeutically effective amount of said immunogenic composition, vaccine or DIVA vaccine.

In any of the aforementioned disclosure of the method or use said feline is selected from a list consisting of: cheetah, puma, jaguar, leopard, lion, lynx, tiger, cat and domestic cat.

In any of the aforementioned disclosure of the method or use said feline is a cat or domestic cat.

In any of the aforementioned disclosure of the method or use said immunogenic composition, vaccine or DIVA vaccine is administered in two or more doses.

In any of the aforementioned disclosure of the method or use said immunogenic composition, vaccine or DIVA vaccine is administered in two doses.

In any of the aforementioned disclosure of the method or use said immunogenic composition, vaccine or DIVA vaccine is administered to the feline from an age of 4, 5 or 6 weeks onwards.

In any of the aforementioned disclosure of the method or use said immunogenic composition, vaccine or DIVA vaccine is administered to the feline from an age of 6 weeks onwards.

In any of the aforementioned disclosure of the method or use said immunogenic composition, vaccine or DIVA vaccine is administered for the first time to the feline within 4 weeks of age to 25 weeks of age.

In any of the aforementioned disclosure of the method or use said immunogenic composition, vaccine or DIVA vaccine is for the first time administered to the feline within 6 weeks of age to 20 weeks of age.

In any of the aforementioned disclosure of the method or use a first dose of the immunogenic composition, vaccine or DIVA vaccine is administered to the feline from an age of 4, 5 or 6 weeks onwards and the second dose is administered 2 to 8 weeks later.

In any of the aforementioned disclosure of the method or use a first dose of the immunogenic composition, vaccine or DIVA vaccine is administered to the feline from an age of 6 weeks onwards and the second dose is administered 2 to 8 weeks later.

In any of the aforementioned disclosure of the method or use after the second (booster) vaccination a further vaccination is done on a yearly basis (an annual booster vaccination).

In any of the aforementioned disclosure of the method or use said immunogenic composition, vaccine or DIVA vaccine is administered intramuscular, intradermal, subcutaneous, orally or nasally.

In any of the aforementioned disclosure of the method or use said immunogenic composition, vaccine or DIVA vaccine comprises 1×10⁴ to 1×10¹⁰ TCIDso of the EHV per ml.

In any of the aforementioned disclosure of the method or use said immunogenic composition, vaccine or DIVA vaccine comprises 1×10⁶ to 1×10⁹ TCIDso of the EHV per ml.

In any of the aforementioned disclosure of the method or use one dose of the immunogenic composition, vaccine or DIVA vaccine comprises 1×10⁴ to 1×10¹⁰ TCIDso of the EHV.

In any of the aforementioned disclosure of the method or use one dose of the immunogenic composition, vaccine or DIVA vaccine comprises 1×10⁶ to 1×10⁹ TCIDso of the EHV.

In any of the aforementioned disclosure of the method or use said method results in an improvement in an efficacy parameter selected from the group consisting of: a reduction in weight loss, a lower virus load in lungs, a reduction in viremia, a reduction in lung lesions, reduced respiratory and alveolar epithelial lesions, a reduced ocular discharge, a reduced conjunctivitis, a reduced rhinitis, a reduced and/or shortened shedding of virus, a reduced rectal temperature, reduced clinical symptoms (in particular respiratory symptoms), increased induction of (neutralizing) anti-FHV virus antibodies, increased stimulation of T-cells against FHV, increased stimulation of B-cells against FHV virus, and a reduction of proinflammatory cytokines, e.g. IL10, in lungs, or combinations thereof, in comparison to a feline of a non-immunized control group of the same species.

In any of the aforementioned disclosure of the method or use said immunogenic composition, vaccine or a DIVA vaccine protects against a homologous and/or heterologous challenge with a FHV.

The disclosure further comprises:
An EHV (Equine Herpesvirus) comprising a FHV gD encoding sequence or a fragment thereof inserted into ORF1/3 and a FHV gB encoding sequence or a fragment thereof inserted into ORF70.

The disclosure further comprises:
An EHV (Equine Herpesvirus) comprising a FHV gD encoding sequence or a fragment thereof inserted into ORF1/3 and a FHV gB encoding sequence or a fragment thereof inserted into ORF70.

The disclosure further comprises:
An EHV (Equine Herpesvirus) comprising:
i) a FHV gD encoding sequence consisting of or comprising a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 200, at least 300 or at least 350 consecutive amino acids, inserted into ORF1/3, and
ii) a FHV gB encoding sequence consisting of or comprising a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 150, at least 200, at least 300, at least 400, at least 500, at least 600, at least 700 or at least 800 consecutive amino acids, inserted into ORF70.

The disclosure further comprises:
An EHV (Equine Herpesvirus) comprising a FHV gD encoding sequence consisting of or comprising a nucleic acid sequence encoding an amino acid sequence as set forth in SEQ ID NO:1 or a sequence having at least 95% sequence identity thereto inserted into ORF1/3 and a FHV gB encoding sequence consisting of or comprising a nucleic acid sequence encoding an amino acid sequence as set forth in SEQ ID NO:2 or a sequence having at least 95% sequence identity thereto inserted into ORF70.

The disclosure further comprises:
An immunogenic composition, vaccine or DIVA vaccine comprising an EHV (Equine Herpesvirus) comprising:
i) a FHV gD encoding sequence consisting of or comprising a nucleic acid sequence encoding an amino acid sequence as set forth in SEQ ID NO:1 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 200, at least 300 or at least 350 consecutive amino acids, inserted into ORF1/3, and
ii) a FHV gB encoding sequence consisting of or comprising a nucleic acid sequence encoding an amino acid sequence as set forth in SEQ ID NO:2 or a sequence having at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto or a fragment thereof having at least 100, at least 150, at least 200, at least 300, at least 400, at least 500, at least 600, at least 700 or at least 800 consecutive amino acids, inserted into ORF70.

The disclosure further comprises:
The disclosed EHV, immunogenic composition, vaccine or DIVA vaccine for therapeutic use.

The disclosure further comprises:
The disclosed EHV, immunogenic composition, vaccine or DIVA vaccine for use as an immunogen or vaccine.

The disclosure further comprises:
The disclosed EHV, immunogenic composition, vaccine or DIVA vaccine for use as a medicament.

The disclosure further comprises:
The disclosed EHV, immunogenic composition, vaccine or DIVA vaccine for the manufacture of a medicament.

The disclosure further comprises:
Use of the disclosed EHV, immunogenic composition, vaccine or DIVA vaccine for the treatment and/or prophylaxis of FHV infections in felines.

The disclosure further comprises:
A method of preparing an EHV (Equine Herpesvirus) comprising a Feline Herpes Virus (FHV) Antigen encoding sequence inserted into ORF70 (US4) and/or ORF1/3 comprising:
i) providing an EHV; and
ii) providing a FHV Antigen encoding sequence; and
iii) inserting said FHV Antigen encoding sequence from step ii) into the ORF70 (US4) and/or ORF1/3 insertion site of the EHV of step i); and
IV) obtaining said EHV comprising a FHV Antigen encoding sequence inserted into ORF70 (US4) and/or ORF1/3.

The disclosure further comprises:
A method of preparing an EHV (Equine Herpesvirus) comprising a Feline Herpes Virus (FHV) Antigen encoding sequence inserted into ORF70 (US4) and/or ORF1/3 comprising:
i) providing an EHV; and
ii) providing a FHV gD, gB or gC encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids; and
iii) inserting said FHV gD, gB or gC encoding sequence from step ii) into the ORF70 (US4) and/or ORF1/3 insertion site of the EHV of step i); and
iv) obtaining said EHV comprising FHV gD, gB or gC encoding sequence inserted into ORF70 (US4) and/or ORF1/3.

In any of the aforementioned disclosure of the method of preparing an EHV said EHV is attenuated.

In any of the aforementioned disclosure of the method of preparing an EHV said EHV is RacH or RacH SE.

The disclosure further comprises:
A method of differentiating felines infected with FHV from felines vaccinated with the disclosed immunogenic composition, vaccine or the DIVA vaccine comprising
   a) obtaining a sample from a feline, and
   b) analyzing said sample in an immuno test, cell culture based assay and/or genomic analytical test.

In any of the aforementioned disclosure of the method of differentiating said immuno test comprises testing whether the sample comprises antibodies specifically recognizing FHV gG.

In any of the aforementioned disclosure of the method of differentiating said feline is infected with FHV if antibodies specifically recognizing FHV gG have been detected.

In any of the aforementioned disclosure of the method of differentiating said genomic analytical test comprises testing whether the sample comprises a FHV gG encoding sequence.

In any of the aforementioned disclosure of the method of differentiating said feline is infected with FHV if a FHV gG encoding sequence has been detected.

In any of the aforementioned disclosure of the method of differentiating said immuno test comprises testing whether the sample comprises antibodies specifically recognizing EHV gD and/or EHV gC.

In any of the aforementioned disclosure of the method of differentiating said feline is vaccinated with the disclosed immunogenic composition, vaccine or DIVA if antibodies specifically recognizing the EHV gD and/or EHV gC have been detected.

In any of the aforementioned disclosure of the method of differentiating said genomic analytical test comprises testing whether the sample comprises a EHV gD and/or EHV gC encoding sequence.

In any of the aforementioned disclosure of the method of differentiating said feline is vaccinated with the disclosed immunogenic composition, vaccine or DIVA vaccine if a EHV gD and/or EHV gC encoding sequence has been detected.

In any of the aforementioned disclosure of the method of differentiating said immuno test is an EIA (enzyme immunoassay) or ELISA (enzyme linked immunosorbent assay), or, wherein the genomic analytical test is a PCR (polymerase chain reaction), RT-PCR (reverse transcriptase polymerase chain reaction) or real time PCR (polymerase chain reaction).

In any of the aforementioned disclosure of the method of differentiating said feline is a cat.

In any of the aforementioned disclosure of the method of differentiating said sample is a serum sample.

In any of the aforementioned disclosure of the method of differentiating said ELISA is an indirect ELISA, Sandwich ELISA, a competitive ELISA or blocking ELISA.

In any of the aforementioned disclosure of the method of differentiating said genomic analytical test is a PCR (polymerase chain reaction), RT-PCR (reverse transcriptase polymerase chain reaction) or real time PCR (polymerase chain reaction).

In any of the aforementioned disclosure of the method of differentiating said cell culture based assay is a CPE (Cytopathic effect or cytopathogenic effect) assay.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic drawing of p1_3-p430-FHVgD(co).

FIG. 2. Schematic drawing of p455-FHVgB(co).

FIG. 3. Schematic drawing of rEHV-1-p430-FHVgD(co)-p455-FHVgB(co).

FIG. 4. Schematic drawing of p1_3-p430-FHVgD(co)F2AgB(co).

FIG. 5. Schematic drawing of rEHV-1-p430-FHVgD(co)F2AgB(co).

FIG. 6. Schematic drawing of p455-FHVgB(n).

FIG. 7. Schematic drawing of rEHV-1-p430-FHVgD(co).

FIG. 8. Schematic drawing of rEHV-1-p455-FHVgB(n).

FIG. 9. Schematic drawing of pFHgB-FHVgB(n).

FIG. 10. Schematic drawing of rEHV-1-p430-FHVgD(co)-pFHgB-FHVgB(n).

FIG. 11. Schematic drawing of p1_3-p430-FHVgD(co)IRESgB(n).

FIG. 12. Schematic drawing of rEHV-1-p430-FHVgD(co)IRESgB(n).

FIG. 13. Swine serology study FHV-1 SN results using geometric mean titer for treatment groups.

FIG. 14. Swine serology study EHV-1 SN results using geometric mean titer for treatment groups.

FIG. 15. Feline challenge study clinical signs results of cats showing moderate to severe signs for >2 days (score>1).

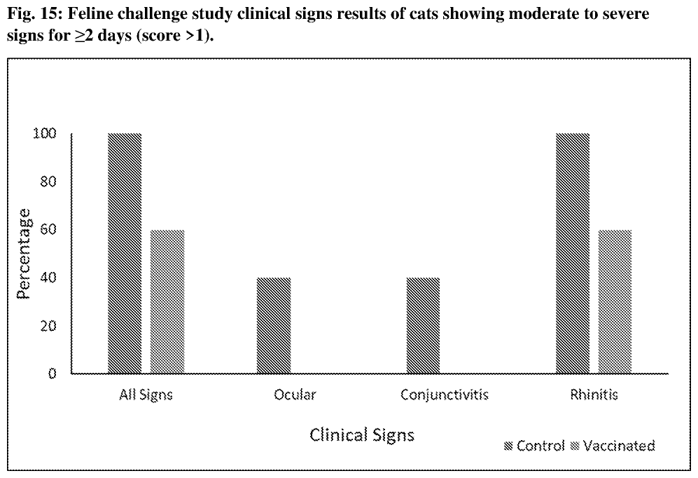

FIG. 16. Feline challenge study average of body weights (kg) by group.

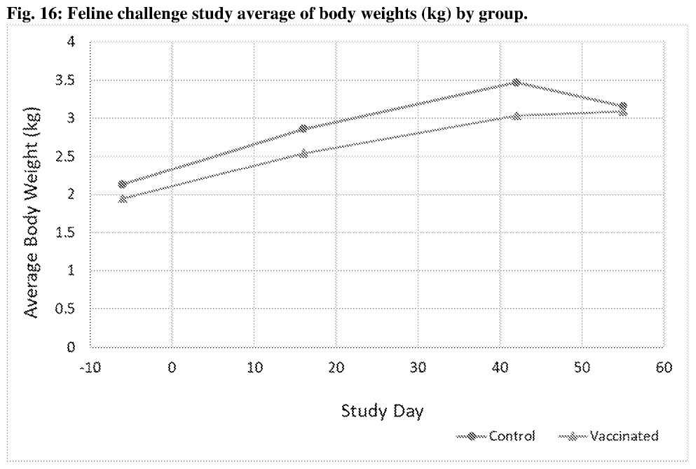

FIG. 17. Feline challenge study vaccinate group only geomean and individual animal EHV-1 SN titers. x=individual 50% endpoint titer of animal within group.

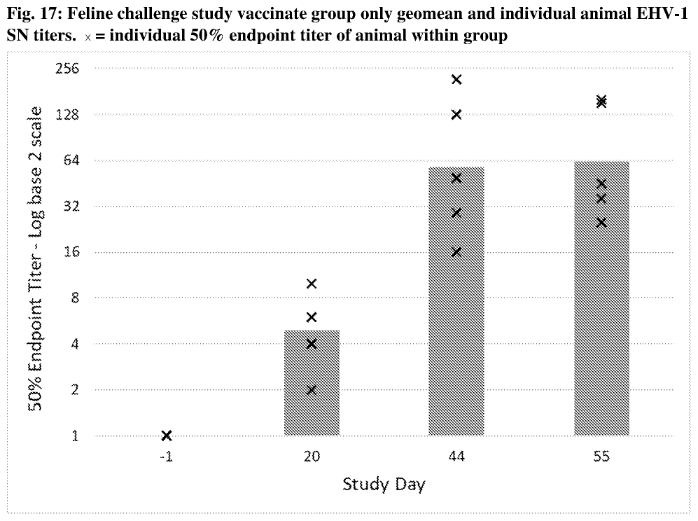

FIG. 18. Feline challenge study group geomean and individual animal FVR SN titers for groups 1 and 2. x=individual value of group 1 animals; ●=individual value of group 2 animals.

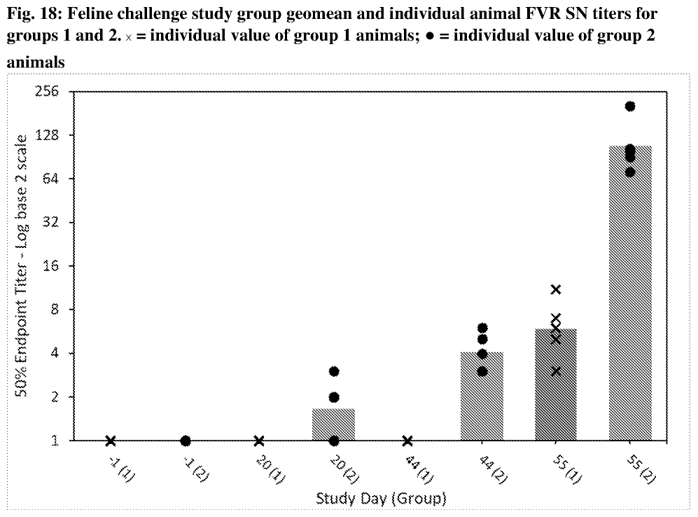

### SEQUENCES OVERVIEW

### The Following Sequences are Detailed and Disclosed Hereby in the Present Invention:

### Glycoproteins:

SEQ ID NO: 1 FHVgD Amino Acid Sequence from Strain C-27
SEQ ID NO: 2 FHVgB Amino Acid Sequence from Strain C-27
SEQ ID NO: 3 FHVgC Amino Acid Sequence from Strain C-27
SEQ ID NO: 4 Codon Optimized FHVgD Nucleotide Sequence from FHV-1 Strain C-27
SEQ ID NO: 5 Native FHVgD Nucleotide Sequence from FHV-1 Strain C-27
SEQ ID NO: 6 Codon Optimized FHVgB Nucleotide Sequence from FHV-1 Strain C-27
SEQ ID NO: 7 Native FHVgB Nucleotide Sequence from Strain C-27
SEQ ID NO: 8 Codon Optimized FHVgC Nucleotide Sequence from FHV-1 Strain C-27
SEQ ID NO: 9 Native FHVgC Nucleotide Sequence from Strain C-27 ORF70 insertion site:
SEQ ID NO: 10 801 bp deletion within ORF70 for the wild-type EHV-1 strain ab4 (Genbank accession number AY665713.1)
SEQ ID NO: 11 801 bp portion within ORF70 (US4) for RacH
SEQ ID NO: 12 left US4 flanking region (417 bp)
SEQ ID NO: 13 right US4 flanking region (431 bp)
SEQ ID NO: 14 left US4 flanking region (417 bp)
SEQ ID NO: 15 right US4 flanking region (431 bp)
SEQ ID NO: 16 left US4 flanking region (283 bp)
SEQ ID NO: 17 right US4 flanking region (144 bp)

### Promoters

SEQ ID NO: 18 p430 promoter
SEQ ID NO: 19 p⁴⁵⁵ promoter
SEQ ID NO: 24 truncated FHV-1 gB Promoter

### Primers:

SEQ ID NO: 20 to SEQ ID NO: 23 Primer

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Generation of Recombinant EHV-1 with FHV-1 Glycoprotein D at ORF1/3 Insertion Site and FHV-1 Glycoprotein B at ORF70 Insertion Site (rEHV-1-p430-FHVgD(Co)-p455-FHVgB(Co))

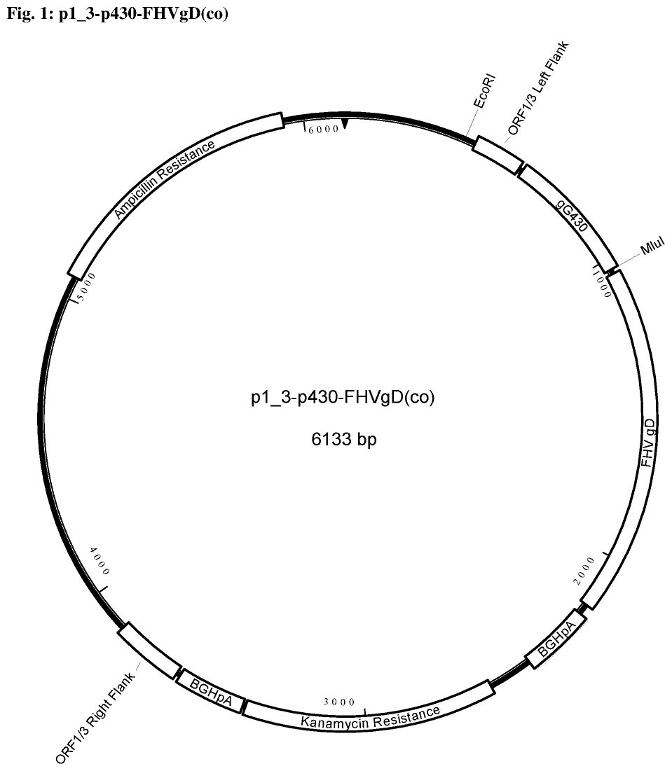

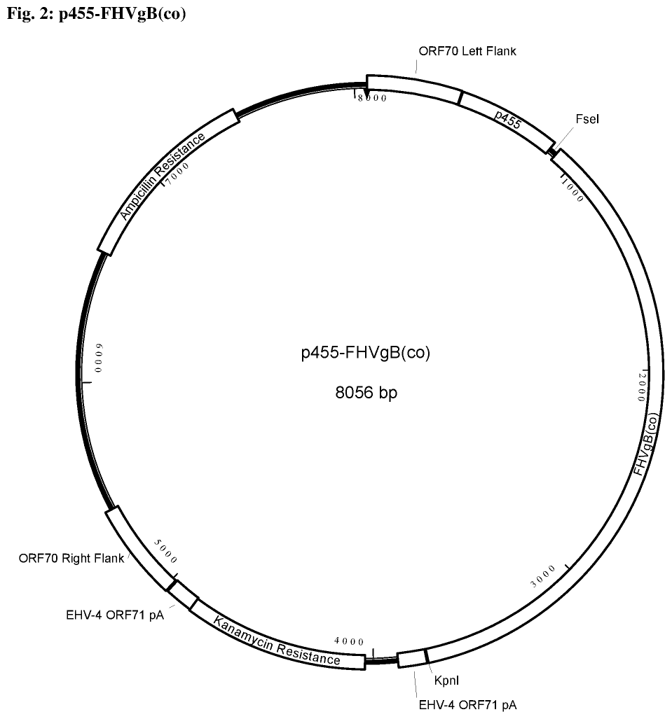

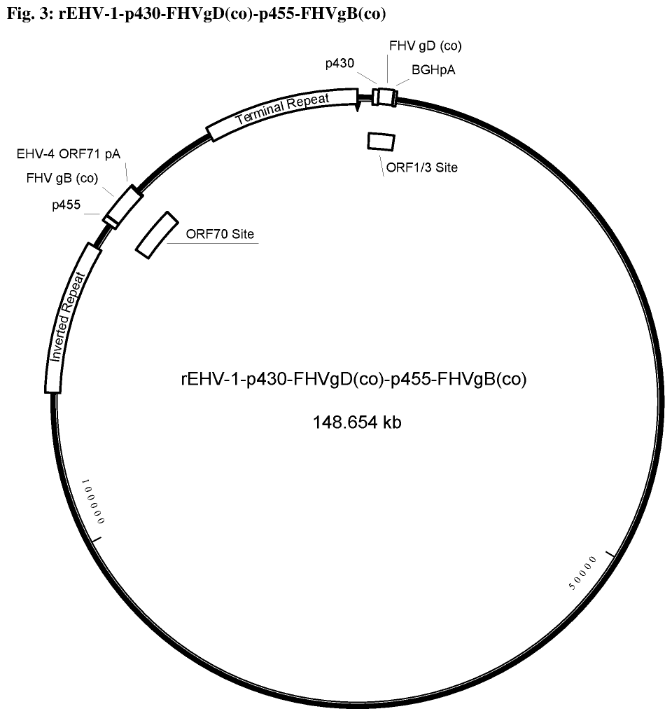

Synthetic, codon-optimized FHVgD coding sequence (SEQ ID NO:4) is digested with restriction endonucleases and ligated into p1_3-mCMV (murine CMV promoter as described by Dorsch-Hasler et al. 1985) vector containing the mCMV promoter and flanks for EHV-1 ORF1/3 homologous recombination, digested with the same restriction endonucleases, resulting in p1_3-mCMV-FHVgD(co). The EHV-4 gG430 promoter p430 (SEQ ID NO:18) is excised from pgG430-MCS1 vector using MluI and EcoRI restriction endonucleases and ligated into p1_3-mCMV-FHVgD(co), resulting in p1_3-p430-FHVgD(co) (FIG. 1). Synthetic, codon-optimized FHVgB coding sequence (SEQ ID NO:6) is digested with FseI and KpnI restriction endonucleases and cloned into the p455 shuttle vector containing the EHV-4 major capsid protein (MCP) promoter p455 (SEQ ID NO:19) and flanks for EHV-1 ORF70 homologous recombination, digested with the same restriction endonucleases, resulting in p455-FHVgB(co) (FIG. 2). To generate a recombinant EHV-1 virus with two FHV-1 antigens at two different insertion sites, the RED recombination system is used to insert codon-optimized FHV-1 gD and FHV-1 gB expression cassettes into ORF1/3 and ORF70 insertion sites, respectively, to generate rEHV-1-p430-FHVgD(co)-p455-FHVgB(co) (FIG. 3). After kanamycin selection, rEHV-1-p430-FHVgD(co)-p455-FHVgB(co) BAC DNA is transfected into Al-ST cells to rescue recombinant virus-rEHV-1-p430-FHVgD(co)-p455-FHVgB(co).

### Example 2

Generation of Recombinant EHV-1 with FHV-1 Glycoprotein D and B at ORF1/3 Insertion Site (rEHV-1-p430-FHVgD(Co)F2AgB(Co))

The Polymerase chain reaction (PCR) is performed on p1_3-mCMV-FHVgD(co) with primers F2A Oligo (Table 1; SEQ ID NO:20) and FHVgD-F(Table 1; SEQ ID NO:21) to amplify a 347 bp fragment including 270 bp of the 3′-end of the FHV gD (co) coding sequence, void of the stop codon, a codon-optimized foot and mouth disease virus (FMDV) 2A peptide sequence (F2A), and 23 bp of the 5′-end of the FHV gB (co) coding sequence.

TABLE 1
Primers for construction of p1_3-mCMV-FHVgD(co)
F2AgB(co)

| Primer | Sequence |
|---|---|
| F2A | CCCAGGTCGCCCCTGGTGCTCATGGGTCCTGGGTTGGACTCC |
| Oligo | ACGTCGCCAGCCAGCTTCAGCAGGTCGAAGTTCAGGGGGTGG |
| | TGGGTGGTGTGGATGA (SEQ ID NO: 20) |
| FHVgD-F | GACTCTAGAGGCGAGTCTTCCGGCC (SEQ ID NO: 21) |
| FHVgB-F | ATGAGCACCAGGGGCGACCTGGG (SEQ ID NO: 22) |
| 3′- | CTATGTCGACCTCGAGGGTACCTTACACCAGGTTGG |
| FHVgB-R | (SEQ ID NO: 23) |

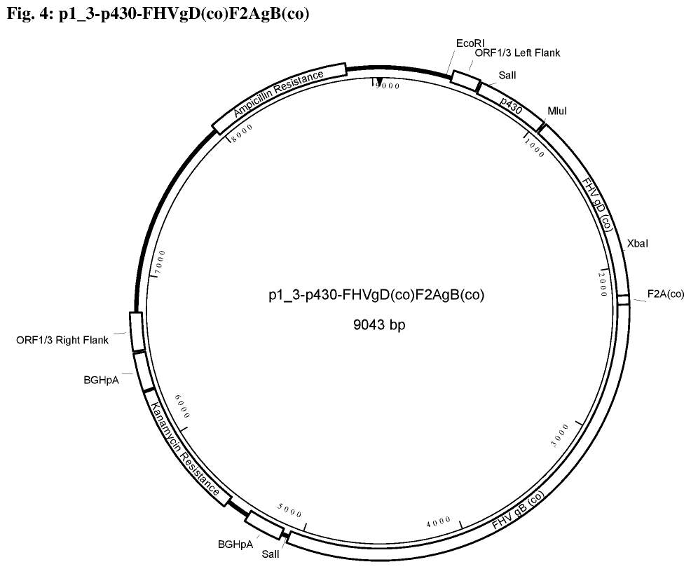

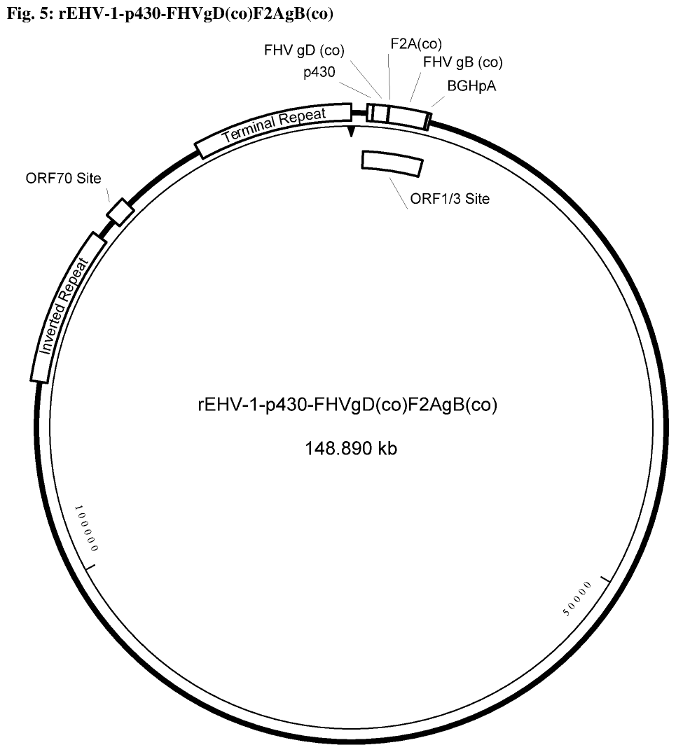

A second PCR is performed with primers FHVgB-F (Table 1; SEQ ID NO:22) and 3′-FHVgB-R (Table 1; SEQ ID NO:23) to amplify the full FHV gB(co) coding sequence from p455-FHVgB(co), 2,487 bp in length. The two PCR fragments are subsequently joined by overlap-extension PCR (OE-PCR), to generate a fragment with XbaI and SalI sites on the 5′ and 3′-ends, respectively. The fused coding sequence is digested with XbaI and SalI restriction endonucleases and cloned into the multiple cloning site 1 (MCS1) of the p1_3-mCMV-FHVgD(co) via XbaI and SalI restriction sites, resulting in p1_3-mCMV-FHVgD(co)F2AgB(co). The EHV-4 gG430 promoter is excised from the pgG430-MCS1 vector using MluI and EcoRI restriction endonucleases and ligated into p1_3-mCMV-FHVgD(co)F2AgB(co) digested with the same restriction endonucleases, resulting in p1_3-p430-FHVgD(co)F2AgB(co) (FIG. 4). By en passant mutagenesis using the RED recombination system (Tischer et al. 2006. Biotechnol. Tech. 40, 191-197) codon-optimized FHV-1 gD and FHV-1 gB expression cassettes are inserted into the ORF1/3 insertion site, linked by an F2A cleavage site, to generate rEHV-1-p430-FHVgD(co)F2AgB(co) (FIG. 5). After kanamycin selection, rEHV-1-p430-FHVgD(co)F2AgB(co) BAC DNA is transfected into Al-ST cells to rescue recombinant virus-rEHV-1-p430-FHVgD(co)F2AgB(co).

### Example 3

Generation of Recombinant EHV-1 with Codon-Optimized FHV-1 Glycoprotein D at ORF1/3 Insertion Site (rEHV-1-p430-FHVgD(Co)) or Native FHV-1 Glycoprotein B at ORF70 Insertion Site (rEHV-1-p455-FHVgB(n))

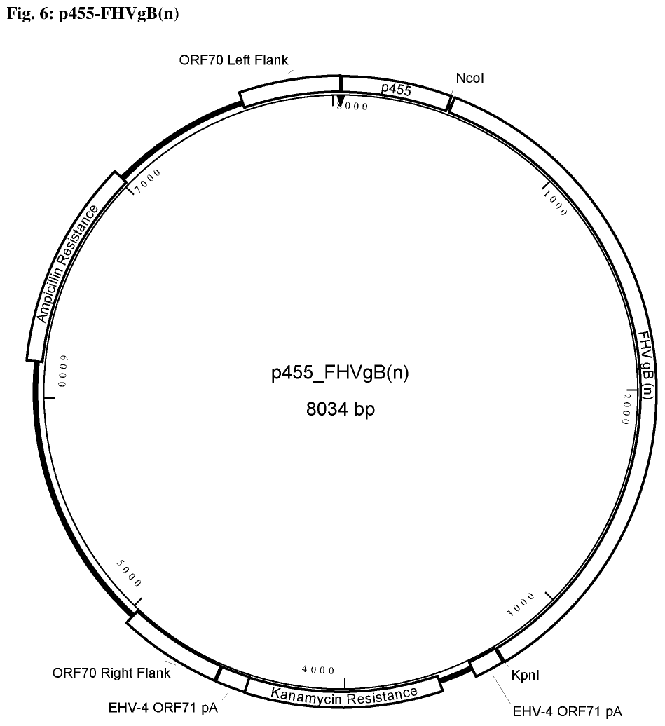

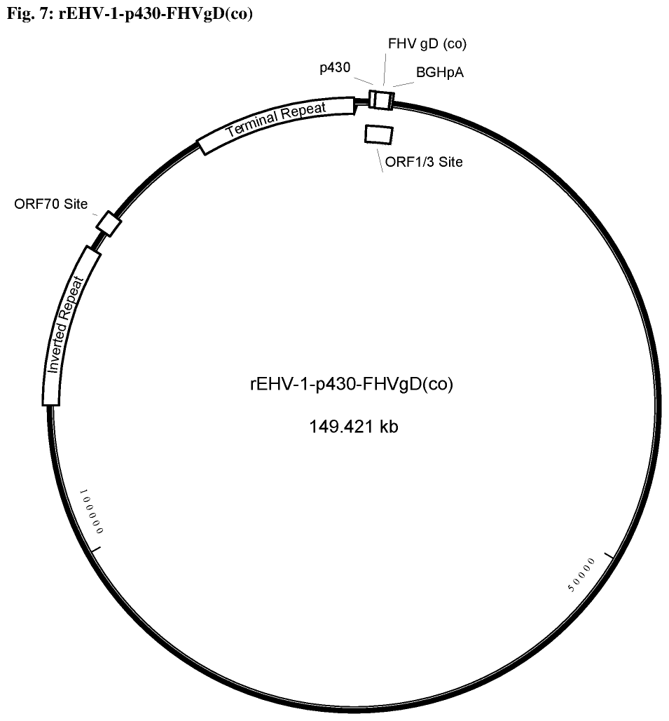

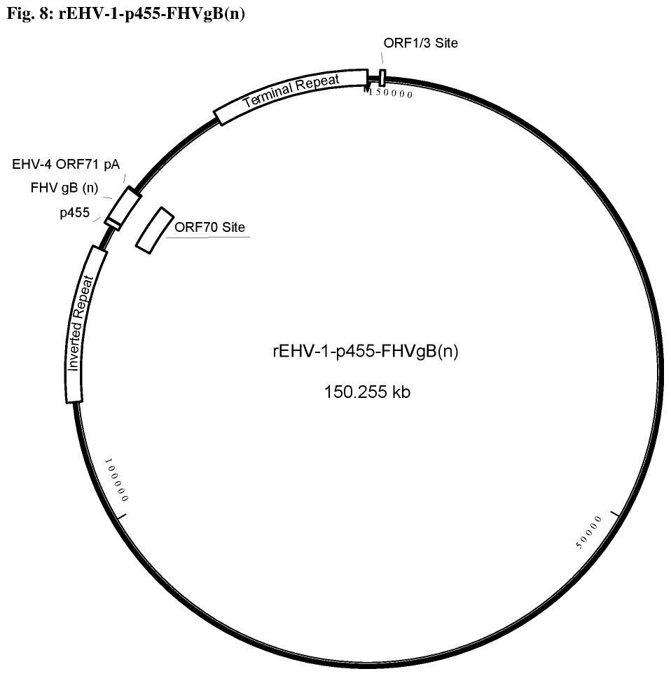

Synthetic, native FHVgB coding sequence (SEQ ID NO:7) is digested with NcoI and KpnI restriction endonucleases and cloned into the p455 plasmid digested with the same restriction endonucleases, resulting in p455-FHVgB(n) (FIG. 6). By en passant mutagenesis using the RED recombination system (Tischer et al. 2006. Biotechnol. Tech. 40, 191-197) codon-optimized FHV-1 gD from p1_3-FHVgD(co) is inserted into the ORF1/3 insertion site (described exemplarily in WO 2018/054837 A1) to generate rEHV-1-p430-FHVgD(co) (FIG. 7) BAC DNA followed by transfection of A1-ST cells to rescue rEHV-1-p430-FHVgD(co) virus. Similarly, en passant mutagenesis is used to insert native FHV-1 gB from p455-FHVgB(n) into the ORF70 insertion site (described exemplarily in WO 2018/054837 A1) to generate rEHV-1-p455-FHVgB(n) (FIG. 8) BAC DNA followed by transfection of Al-ST cells to rescue rEHV-1-p455-FHVgB(n) virus.

### Example 4

Generation of Recombinant EHV-1 with Codon-Optimized FHV-1 Glycoprotein D at ORF1/3 Insertion Site with p430 Promoter and Native FHV-1 Glycoprotein B at ORF70 Insertion Site with Native FHV-1 gB Promoter (rEHV-1-p430-FHVgD(Co)-pFHgB-FHVgB(n)) and Generation of Recombinant EHV-1 with FHV-1 Glycoprotein D and B at ORF1/3 (rEHV-1-p430-FHVgD(Co)IRESgB(n))

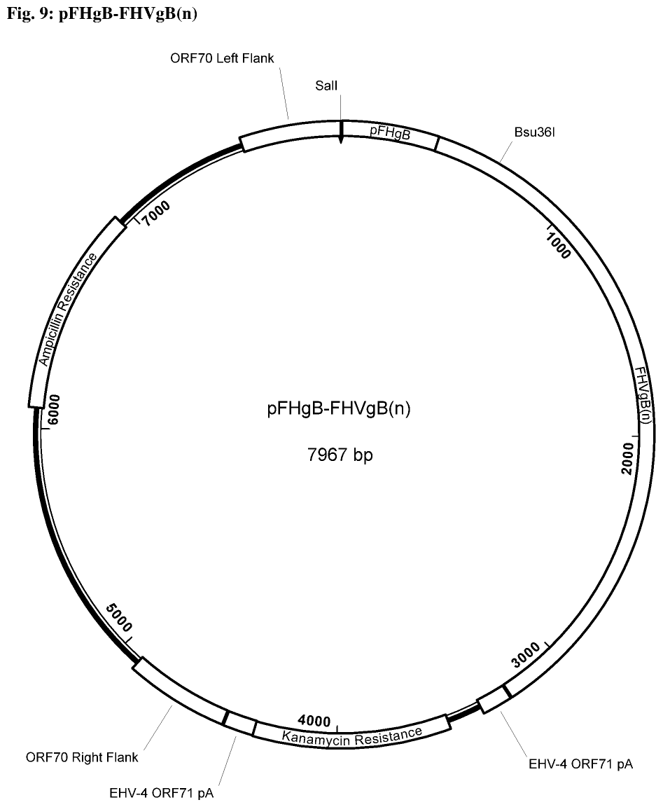

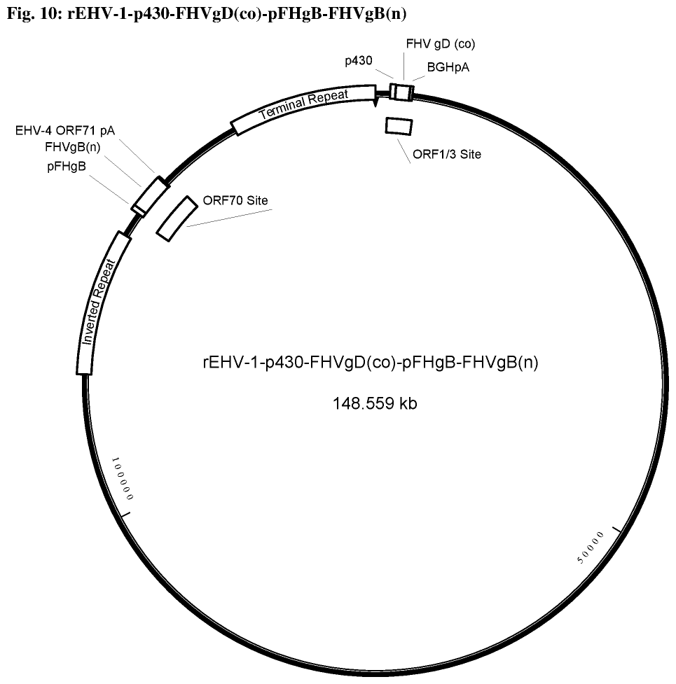

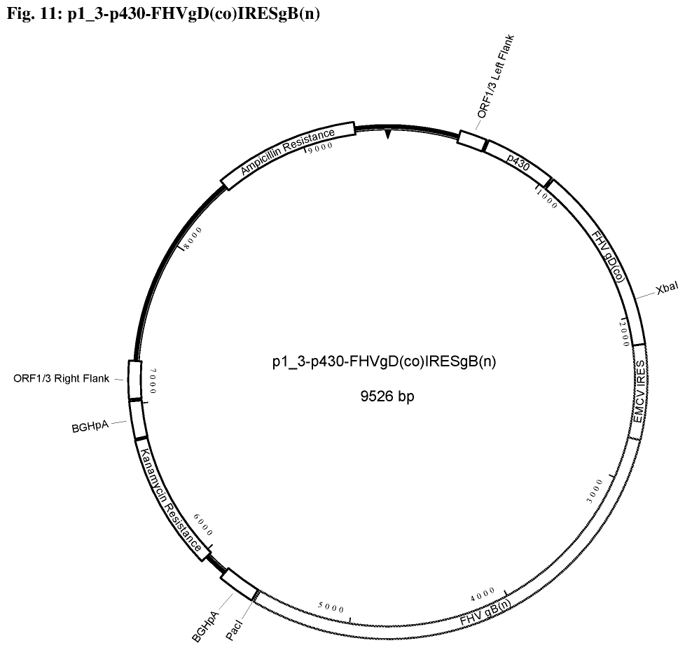

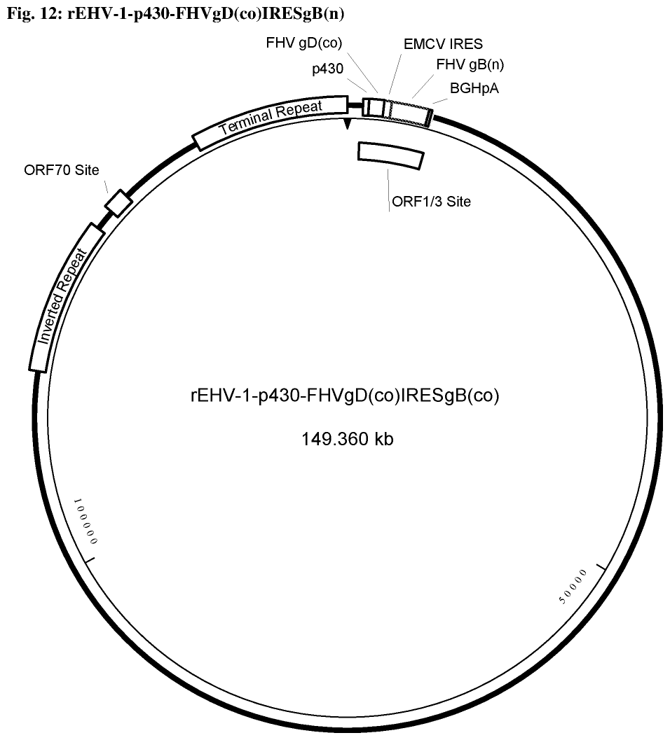

Synthetic 671 bp fragment containing the native promoter for FHV gB (SEQ ID NO:24) is generated by digestion with Bsu36I and SalI restriction endonucleases and ligated into p455-FHVgB(n) digested with the same restriction endonucleases, resulting in pFHgB-FHVgB(n) (FIG. 9). By en passant mutagenesis using the RED recombination system (Tischer et al. 2006. Biotechnol. Tech. 40, 191-197) codon-optimized FHV-1 gD from p1_3-p430-FHVgD(co) is inserted into the ORF1/3 insertion site (described exemplarily in WO 2018/054837 A1) followed by RED recombination to insert native FHV-1 gB driven by a native FHV-1 gB promoter into the ORF70 insertion site (described exemplarily in WO 2018/054837 A1) to generate rEHV-1-p430-FHVgD(co)-pFHgB-FHVgB(n) (FIG. 10) BAC DNA. This BAC DNA is transfected into Al-ST cells to rescue rEHV-1-p430-FHVgD(co)-pFHgB-FHVgB(n) virus. Synthetic 3,687 bp fragment containing the 3′-end of codon-optimized FHV-1 gD, EMCV IRES, and native FHV-1 gB coding sequence is digested with PacI and XbaI restriction endonucleases and ligated into p1_3-p430-FHVgD(co) digested with the same restriction endonucleases, resulting in p1_3-p430-FHVgD(co)IRESgB(n) (FIG. 11). By en passant mutagenesis using the RED recombination system (Tischer et al. 2006. Biotechnol. Tech. 40, 191-197) codon-optimized FHV-1 gD and native FHV-1 gB expression cassettes are inserted into the ORF1/3 insertion site, linked by an EMCV IRES, to generate rEHV-1-p430-FHVgD(co)IRESgB(n) (FIG. 12) BAC DNA. This BAC DNA is transfected into Al-ST cells to rescue rEHV-1-p430-FHVgD(co)IRESgB(n) virus.

### Example 5

Recombinant EHV-1 Viruses Expressing FHV-1 Glycoproteins D and/or B

Limiting dilutions are performed on Al-ST cells with rEHV-1-p430-FHVgD(co)-p455-FHVgB(co) and rEHV-1-p430-FHVgD(co)F2AgB(co) to remove GFP marker from the virus and obtain a clonal virus population. Correct insertion of the expression cassette into the EHV-1 RacH bacmid backbone is verified by next generation (NextGen) sequencing. Expression of the transgenes in infected cells is analyzed by immunofluorescence assay (IFA). Peak titers determined as TCID50/mL in Al-ST cells are in the same range as titers of the parental virus rEHV-1 RacH, which indicates that transgene expression had no detrimental effect on viral replication (not shown).

In Vitro Characterization of rEHV-1 FHVgD/FHVgB Viruses—IFA

There is no noticeable difference in the plaque size, growth kinetics or titer of the recombinant viruses compared to the parent EHV-1 RacH strain. Expression of FHVgD and FHVgB by rEHV-p430-FHVgD(co)-p455-FHVgB(co) and rEHV-p430-FHVgD(co)F2AgB(co) viruses is assessed by IFA of Al-ST cells infected with EHV-1 RacH, FHV-1 strain F2, rEHV-p430-FHVgD(co)-p455-FHVgB(co) and rEHV-p430-FHVgD(co)F2AgB(co). Cells are stained with mouse anti-FHVgD monoclonal antibody clone 215C1M, mouse anti-FHVgB monoclonal antibody clone 218E4S, cat anti-FVR serum and mouse anti-EHV-1 monoclonal antibody clone 16H9, (all owned by Boehringer Ingelheim). Alexa Fluor 594 goat anti-mouse IgG (Life Technologies, Carlsbad, CA) is used as the secondary antibody for monoclonal primary antibodies and FITC-conjugated goat anti-cat IgG (Jackson ImmunoResearch, West Grove, PA) is used as the secondary for the cat anti-FVR serum. As expected, FHVgD and FHVgB expression is only detected in Al-ST cells infected with the recombinant viruses (Table 2). Staining for FHV gD expression in cells infected with rEHV-p430-FHVgD(co)F2AgB(co) is weaker than the staining for FHV gD expression in cells infected with rEHV-p430-FHVgD(co)-p455-FHVgB(co). Expression of FHVgD and FHVgB by rEHV-p430-FHVgD(co) and rEHV-p455-FHVgB(n) viruses is assessed by IFA of Al-ST cells infected with rEHV-p430-FHVgD(co) and rEHV-p455-FHVgB(n). Cells are stained with mouse anti-FHVgD monoclonal antibody clone 215C1M (owned by Boehringer Ingelheim), mouse anti-FHVgB monoclonal antibody clone 218E4S (owned by Boehringer Ingelheim) and FITC-conjugated anti-EHV-1 ready to use caprine polyclonal antiserum (VMRD, Pullman, WA). Alexa Fluor 594 goat anti-mouse IgG (Life Technologies, Carlsbad, CA) is used as the secondary antibody for monoclonal primary antibodies. As expected, FHV gD expression is only detected in Al-ST cells infected with rEHV-1-p430-FHVgD(co) and FHV gB expression is only detected in Al-ST cells infected with rEHV-1-p455-FHVgB(n) (Table 2).

TABLE 2
Expression of FHV gD and FHV gB in A1-ST cells - IFA Results

| Virus | FHV gD | FHV gB | EHV-1 |
|---|---|---|---|
| rEHV-1-p430-FHVgD(co)- | + | W+ | + |
| p455-FHVgB(co) | | | |
| rEHV-1-p430-FHVgD(co)F2AgB(co) | W+ | + | + |
| rEHV-1-p430-FHVgD(co) | + | N | + |
| rEHV-1-p455-FHVgB(n) | N | W+ | + |
| EHV-1 RacH | N | N | + |
| FHV-1 Strain F2 | N | N | N |

N = Negative;
W+ = Weak Staining;
+ = Average Staining

### Conclusion Example 4

Constructs containing FHV gD and FHV gB antigens show expression of both proteins. The FHV gD monovalent construct shows expression of FHV gD and the FHV gB monovalent construct shows expression of FHV gB.

### Example 6

### Testing of Recombinant EHV-1 Vectored Vaccine Expressing FHV-1 gD and gB In Vivo in Swine

To assess serological response of a recombinant EHV-1 vaccine expressing FHV-1 gD and FHV-1 gB, a serology study is performed in swine. A total of eight pigs, approximately 6-7 weeks of age, are randomized into three treatment groups (Table 3).

TABLE 3
Swine serology study design

| | | | Vx Regimen | |
|---|---|---|---|---|
| | | # of | D 0 | Study |
| Group | Treatment | Animals | & D 14 | End |
| T01 | rEHV-gG430-FHVgD(co)- | 3 | 2 mL IM & | D 28 |
| | MCP-FHVgB(co) | | 2 mL IN | |
| T02 | rEHV-gG430- | 3 | 2 mL IM & | |
| | FHVgD(co)F2AgB(co) | | 2 mL IN | |
| T03 | rEHV-1 with non-FHV | 2 | 2 mL IM & | |
| | antigens | | 2 mL IN | |

The treatments consist of two experimental vaccines, rEHV-p430-FHVgD(co)-p455-FHVgB(co) (Group 1; 3 pigs, Titer: 8.02 TCID₅₀/mL) and rEHV-p430-FHVgD(co)F2AgB(co) (Group 2; 3 pigs, Titer: 8.30 TCID₅₀/mL), and a recombinant EHV-1 virus with non-FHV antigens as the negative control (Group 3; 2 pigs, Titer: 7.80 TCID₅₀/mL). On day 0, pigs are administered a 2 mL intramuscular (IM) and a 2 mL intranasal (IN) dose of the appropriate vaccine. On day 14, all animals receive a booster administration of 2 mL IM and 2 mL IN of the appropriate vaccine. For the study schedule refer to Table 4.

TABLE 4
Swine serology study schedule of events

| Study Day | Study Event |
|---|---|
| D 0 | Animal Health Examination |
| | Blood Collection (pre-1st vx) |
| | 1st vaccination |
| D 7 | Blood Collection |
| D 14 | Animal Health Examination |
| | Blood Collection (pre-2nd vx) |
| | 2nd vaccination |
| D 21 | Blood Collection |
| D 28 | Blood Collection |
| | Necropsy and Terminal Serum |

All serum samples are sent to the Animal Health Diagnostic Center (AHDC) at Cornell University (Ithaca, NY) for CPE-based FHV-1 and EHV-1 serum neutralizing (SN) assays.

Group 1 animals vaccinated with rEHV-gG430-FHVgD(co)-MCP-FHVgB(co) show higher FHV-1 serum neutralizing antibodies compared to Group 2 and Group 3 animals at D21 and D28, which are vaccinated with rEHV-gG430-FHVgD(co)F2AgB(co) and a non-relevant rEHV-1 virus, respectively (Table 5 and FIG. 9). Two pigs from Group 1 achieve FHV-1 SN titers>16 following the booster vaccination.

TABLE 5
Swine serology study FHV-1 SN results

| | D | D | D | D | D |
|---|---|---|---|---|---|
| Group | 0 | 7 | 14 | 21 | 28 |
| 1 | <2 | <4 | <2 | 16 | 12 |
| (rEHV-p430-FHVgD(co)-p455-FHVgB(co)) | <2 | <2 | 3 | 12 | 12 |
| | <8 | 3 | 2 | 24 | 16 |
| 2 | <4 | <4 | <4 | 12 | <4 |
| (rEHV-p430-FHVgD(co)F2AgB(co)) | <2 | <2 | <4 | <4 | 4 |
| | <4 | 2 | <2 | 6 | <4 |
| 3 | <2 | <8 | <2 | <2 | <2 |
| (rEHV-1 with non-FHV antigens) | <2 | <2 | <2 | <4 | <4 |

To demonstrate that the study is valid and EHV-1 serum neutralizing antibodies are generated in all animals in all groups, EHV-1 SN tests are performed on serum samples from all animals for all collection time points. All animals from the study show EHV-1 SN titers>16 after the second vaccination (Table 6 and FIG. 10).

TABLE 6
Swine serology study EHV-1 SN results

| | D | D | D | D | D |
|---|---|---|---|---|---|
| Group | 0 | 7 | 14 | 21 | 28 |
| 1 | <8 | <8 | <4 | 48 | 24 |
| (rEHV-p430-FHVgD(co)-p455-FHVgB(co)) | <8 | <4 | <4 | 32 | 32 |
| | <8 | 4 | <4 | 24 | 16 |
| 2 | <8 | <8 | <8 | 32 | 32 |
| (rEHV-p430-FHVgD(co)F2AgB(co)) | <8 | 4 | <4 | 96 | 48 |
| | <4 | <4 | <8 | 32 | 64 |
| 3 | <8 | <8 | <4 | 24 | 32 |
| (rEHV-1 with non-FHV antigens) | <4 | <4 | <4 | 32 | 24 |

To further determine the presence of FHVgD and FHVgB specific antibodies, an HI is performed to check for FHVgD antibodies and an IFA is done to check for FHVgD and FHVgB specific antibodies.

### HI (Hemagglutination Inhibition) Assay

Because FHV gD is known to cause hemagglutination of feline RBCs (Maeda et al. 1998. J Vet Med Sci; 60:881-888), an HI assay is performed on the study serum samples to test for the presence of FHVgD specific antibodies. Hemagglutination antigen is FHVgD from Al-ST cells infected with rEHV-p430-FHVgD(co)-p455-FHVgB(co) and lysed with 0.1% CHAPS. Pigs inoculated with rEHV-p430-FHVgD(co)-p455-FHVgB(co) show two- to four-fold higher HI titers than pigs inoculated with rEHV-p430-FHVgD(co)F2AgB(co) after the booster vaccination on D14, indicating a higher concentration of antibodies specific for FHVgD (Table 7). Serum from animals in group 3 show no HI activity throughout the entirety of the study.

TABLE 7
Swine serology study FHV-1 HI results

| | D | D | D | D | D |
|---|---|---|---|---|---|
| Group | 0 | 7 | 14 | 21 | 28 |
| 1 | <10 | 10 | 10 | 80 | 80 |
| (rEHV-p430-FHVgD(co)- | <10 | 10 | 20 | 160 | 160 |
| p455-FHVgB(co)) | <10 | <10 | 10 | 160 | 160 |
| 2 | <10 | <10 | <10 | 40 | 40 |
| (rEHV-p430-FHVgD(co)F2AgB(co)) | <10 | <10 | <10 | 20 | 20 |
| | <10 | <10 | <10 | 40 | 40 |
| 3 | <10 | <10 | <10 | <10 | <10 |
| (rEHV-1 with non-FHV antigens) | <10 | <10 | <10 | <10 | <10 |

### IFA (Immunofluorescence Assay)

To test study serum for FHVgD and FHVgB specific antibodies Al-ST cells are transfected separately with shuttle vectors p1_3-p430-FHVgD(co) and p455-FHVgB(co). A third set of cells are not transfected for use as negative cell controls. Table 8 shows the results of this assay.

TABLE 8
Swine serology study serum IFA results

| | Anti-FHVgD | | | | | Anti-FHVgB | | | | |
|---|---|---|---|---|---|---|---|---|---|---|
| Group | D 0 | D 7 | D 14 | D 21 | D 28 | D 0 | D 7 | D 14 | D 21 | D 28 |
| 1 | N | W+ | + | + | + | N | N | N | N | W+ |
| | N | W+ | N | S+ | S+ | N | N | W+ | W+ | W+ |
| | N | W+ | W+ | S+ | S+ | N | W+ | W+ | N | W+ |
| 2 | N | W+ | W+ | + | + | N | W+ | N | W+ | W+ |
| | N | W+ | W+ | + | + | N | W+ | N | N | W+ |
| | N | W+ | W+ | + | + | N | W+ | W+ | W+ | W+ |
| 3 | N | W+ | W+ | N | N | N | W+ | W+ | N | N |
| | N | W+ | N | W+ | W+ | N | W+ | W+ | N | N |

N = Negative;
W+ = Weak Staining;
+ = Average Staining;
S+ = Strong Staining

Complementing the HI titers observed from group 1 pigs, these same serum samples also show strong IFA staining on cells transfected with the FHV gD shuttle vector, with even more robust IFA staining evident after the second vaccination on D14. In contrast, the same group 1 sera only weakly stain cells transfected with the FHV gB shuttle vector. Sera from group 2 pigs have low HI titers directed against gD, the same group 2 serum samples are found to moderately stain cells transfected with the FHV gD shuttle vector. Similar to the anti-gB IFA response observed with sera from the group 1 pigs, the serum samples from group 2 pigs only weakly stain cells transfected with the FHV gB shuttle vector, although the staining is punctate. Serum samples from group 3 pigs, inoculated with the non-relevant rEHV-1 virus show only negative or weak non-punctate staining on cells transfected with either FHV gD or FHV gB shuttle vectors at an intensity that is attributed to background staining. The observed background staining from group 3 pigs is considered as an overall baseline for negative IFA reactivity for pigs in groups 1 and 2.

### Conclusion Example 5

Taken together, both constructs work and show FHV-1 serum neutralizing antibodies, HI titers and IFA staining.

### Example 7

### Testing of Recombinant EHV-1 Vectored Vaccine Expressing FHV-1 gD and gB In Vivo in Cats

To assess serological response of a recombinant EHV-1 vaccine expressing FHV-1 gD and FHV-1 gB, a challenge study is performed in the target species, cats.

A total of ten cats, approximately 7-9 weeks of age on day 0, are randomized into two treatment groups (Table 9).

TABLE 9
Feline challenge study design

| | | | | | Challenge |
|---|---|---|---|---|---|
| | # of | | | Challenge | Route of |
| Group | Animals | Vaccine | Challenge | Target Dose | Administration |
| 1 | 5 | N/A | FVR 96-13 | 6.5 | Oral/Nasal |
| | | | | TCID50/mL | |
| 2 | 5 | rEHV-p430-FHVgD(co)- | FVR 96-13 | 6.5 | Oral/Nasal |
| | | p455-FHVgB(co) | | TCID50/mL | |

The treatments consist of five control cats in group 1 to be challenged on day 44 with Feline Herpesvirus Type 1 also known as Feline Rhinotracheitis Virus (FVR) 96-13 (CVB BUA No. 2019020, Titer: 6.50 TCID₅₀/mL), and five cats in group 2 to be vaccinated subcutaneously on day 0 and day 21 with rEHV-p430-FHVgD(co)-p455-FHVgB(co) (Titer: 7.86 TCID₅₀/mL) and subsequently challenged on day 44 with FVR 96-13 (CVB BUA No. 2019020, Titer: 6.50 TCID₅₀/mL). On day 0, group 2 cats are administered a 0.5 mL dose of vaccine subcutaneously, followed by a booster vaccination of the same volume and route of administration on study day 21. On day 44, all cats in the study are challenged with 1 mL of challenge virus administered in equal portions between the intranasal and oropharyngeal routes. For the study schedule refer to Table 10.

TABLE 10
Feline challenge study schedule of events

| Study Day | Activity |
|---|---|
| −7 to −1 | Acclimation |
| | Oral-nasal swabs |
| | Obtain body weights |
| | Blood collection |
| 0 | Vaccination 1 (Group 2 Only) |
| 1-5 | Injection site reactions (Group 2 Only) |
| 20 | Blood Collection |
| | Vaccination 2 (Group 2 Only) |
| 21-26 | Injection site reactions (Group 2 Only) |
| 40 | Body weights |
| | Clinical Observations |
| | Rectal Temperatures |
| 42 | Clinical Observations |
| | Rectal Temperatures |
| 44 | Blood Collection |
| | Challenge |
| 45-50 | Clinical Observations |
| | Rectal Temperatures |
| 50-54 | Clinical Observations |
| | Rectal temperatures in febrile cats until resolution |
| 55 | Body weights |
| | Blood collection |
| | Terminate Study |

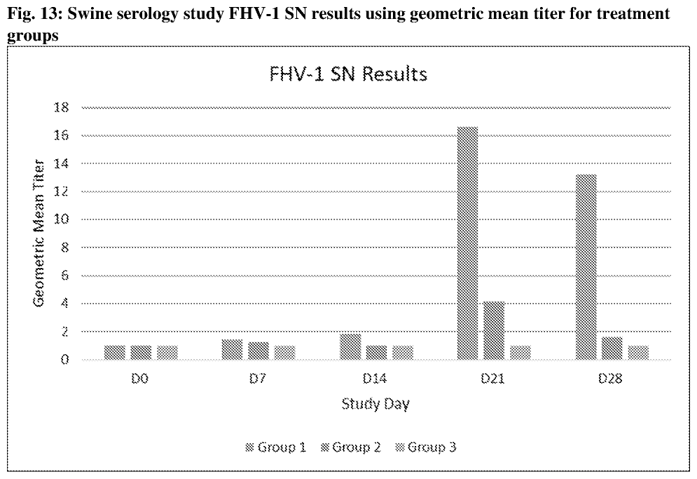

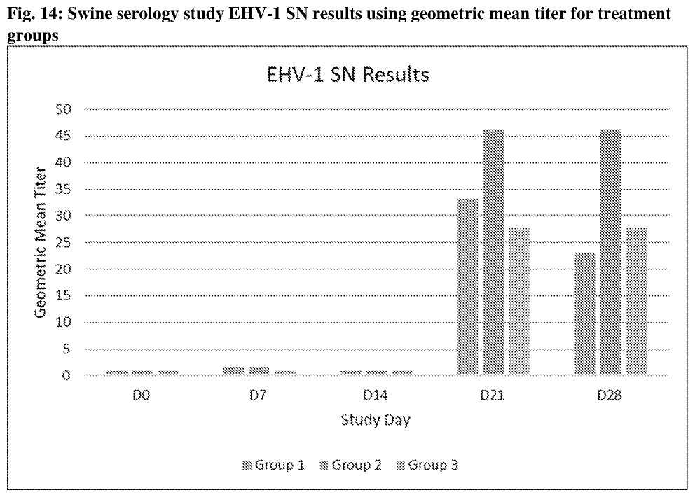

The primary variable is clinical signs of disease and the secondary variable is fever, weight loss and serology. Acceptance criteria for the primary variable includes two or more days of clinical signs post-challenge in non-vaccinate controls to meet case definition and >80% of controls must meet case definition for successful challenge. Acceptance criteria for the secondary variable includes reduction in fever in vaccinated animals compared to non-vaccinated controls, cats must remain seronegative prior to vaccination and show seroconversion post-vaccination and control cats must remain seronegative until challenge. Clinical observations are performed daily post challenge. Cats are weighed before the study, prior to challenge and at the end of the study to determine weight loss. Rectal temperatures are obtained twice prior to challenge for a baseline and daily for the first seven days post-challenge in all cats and then in cats>39.5° C. until resolution. All swabs collected prior to challenge are submitted to University Georgia Athens Veterinary Diagnostic Laboratory (UGA VDL) for screening using a feline respiratory panel PCR. Serum samples from the study are tested for FHV-1 and EHV-1 antibody titers for serum neutralization using a CPE-based readout. All cats in control group have moderate to severe clinical signs for >2 days (FIG. 11). Rhinitis is the most common clinical sign noted among all groups. Ocular and conjunctivitis are not observed in the vaccinated group. Four out of five animals in group 1 show other clinical signs such as audible rales, open-mouth breathing and blood on nostrils from sneezing. No vaccinates show these findings. No local or systemic adverse events are noted in the vaccinated cats. Body weight of the control cats drop post challenge while the vaccinated group remains the same or has slight weight gain (FIG. 12). Three cats in the control group have a fever at four days post challenge, where none of the vaccinated animals has a fever (data not shown). All vaccinated animals develop EHV-1 serum neutralization titers after the first vaccination and a booster response is seen after the second vaccination (FIG. 13). The challenge control group is seronegative for EHV-1 neutralizing antibodies throughout the duration of the study (data not shown). All vaccinated animals develop FHV-1 serum neutralization titers after the second vaccination (FIG. 14). Vaccination induces a good priming of the neutralizing antibody response, as shown by the spike in serum neutralization titers in vaccinated animals post-challenge (day 55).

### Conclusion Example 6

Overall, vaccination with rEHV-p430-FHVgD(co)-p455-FHVgB(co) results in a reduction in severity and duration of clinical signs of disease compared to non-vaccinated control animals. Furthermore, animals vaccinated with rEHV-p430-FHVgD(co)-p455-FHVgB(co) have higher FHV-1 serum neutralization titers post-challenge, indicating a positive vaccine effect.

1. An EHV (Equine Herpesvirus) comprising a Feline Herpes Virus (FHV) Antigen encoding sequence inserted into ORF70 (US4) and/or ORF1/3.

2. An EHV (Equine Herpesvirus) comprising one single Feline Herpes Virus (FHV) Antigen encoding sequence inserted into ORF70 (US4) and/or one single Feline Herpes Virus (FHV) Antigen encoding sequence inserted into ORF1/3.

3. The EHV according to claim 1, wherein the FHV Antigen encoding sequence encodes equal or less than 1400 amino acids.

4. The EHV according to claim 1, wherein the FHV Antigen encoding sequence is one FHV Antigen encoding sequence.

5. The EHV according to claim 1, wherein the FHV Antigen encoding sequence is a glycoprotein encoding sequence or a fragment thereof.

6. The EHV according to claim 1, wherein the FHV Antigen encoding sequence is a FHV gD (glycoprotein D) encoding sequence or a fragment thereof and/or a FHV gB (glycoprotein B) encoding sequence or a fragment thereof and/or a FHV gC (glycoprotein C) encoding sequence or a fragment thereof.

7. The EHV according to claim 1, wherein the FHV Antigen encoding sequence is a gD, gB or gC encoding sequence consisting of or comprising a nucleic acid sequence encoding an amino acid sequence as set forth in SEQ ID NO:1, SEQ ID NO:2 or SEQ ID NO:3 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 100, at least 200, at least 300 or at least 350 consecutive amino acids.

8. The EHV according to claim 1, wherein the FHV Antigen encoding sequence is a gD, gB or gC consisting of or comprising a nucleic acid sequence as set forth in SEQ ID NO:4, SEQ ID NO:5, SEQ ID NO:6, SEQ ID NO:7, SEQ ID NO:8 or SEQ ID NO:9 or a sequence having at least 70%, at least 75%, at least 80%, at least 85%, at least 90%, at least 93%, at least 95%, at least 96%, at least 97%, at least 98%, at least 99%, at least 99.5%, at least 99.6%, at least 99.7%, at least 99.8%, at least 99.9%, at least 99.95%, at least 99.98% or at least 99.99% sequence identity thereto, or a fragment thereof having at least 300, at least 600, at least 900 or at least 1050 consecutive nucleic acids.

9. The EHV according to claim 1, wherein the EHV is attenuated and/or recombinant.

10. The EHV according to claim 1, wherein the EHV vector is EHV-1 and/or RacH or RacH SE.

11. An immunogenic composition, vaccine or DIVA vaccine comprising an EHV according to claim 1.

12. The immunogenic composition, vaccine or DIVA vaccine according to claim 11, wherein the immunogenic composition, vaccine or DIVA vaccine further comprises a pharmaceutically acceptable carrier.

13. A method for immunizing a feline comprising administering to such feline an immunogenic composition, vaccine or a DIVA vaccine according to claim 11.

14. A method for the treatment or prophylaxis of clinical signs caused by FHV in felines, in comparison to a feline of a non-immunized control group, comprising administering to such feline a therapeutically effective amount of an immunogenic composition, vaccine or DIVA vaccine according to claim 11.

15. A method for the treatment or prophylaxis of respiratory disease caused by FHV in felines, in comparison to a feline of a non-immunized control group, comprising administering to such feline a therapeutically effective amount of an immunogenic composition, vaccine or DIVA vaccine according to claim 11.

16. The method according to claim 13, wherein the immunogenic composition, vaccine or DIVA vaccine is administered in two or more doses.

17. The method according to claim 13, wherein said immunogenic composition, vaccine or DIVA vaccine is administered intramuscular, intradermal, subcutaneous, orally or nasally.

18. The EHV according to claim 2, wherein the FHV Antigen encoding sequence encodes equal or less than 1400 amino acids.

19. The EHV according to claim 3, wherein the FHV Antigen encoding sequence is one FHV Antigen encoding sequence.
